(19) United States
(12) Patent Application Publication
(10) Pub. No.: US 2017/0235917 A1
(43) Pub. Date: Aug. 17, 2017

(54) SYSTEMS AND METHODS FOR MONITORING AND CONTROLLING A CARDIOVASCULAR STATE OF A SUBJECT

(71) Applicant: THE GENERAL HOSPITAL CORPORATION, Boston, MA (US)

(72) Inventors: Ramin Bighamian, Boston, MA (US); Jin-Oh Hahn, Boston, MA (US); Andrew T. Reisner, Boston, MA (US); Bryce Yapps, Boston, MA (US)

(21) Appl. No.: 15/501,972

(22) PCT Filed: Aug. 7, 2015
(86) PCT No.: PCT/US15/44350

§ 371 Date: Feb. 6, 2017

Related U.S. Application Data
(60) Provisional application No. 62/035,177, filed on Aug. 8, 2014.

(51) Int. Cl. G06F 19/00; A61B 5/021; A61B 5/00

(52) U.S. Cl. CPC G06F 19/3456; G06F 19/3418; G06F 19/322; G06F 19/3487; G06F 19/3437; A61B 5/742; A61B 5/021; G06F 19/3431; A61B 5/024

(57) ABSTRACT

Systems and methods for monitoring and/or controlling a cardiovascular system of a subject are disclosed. In some aspects, the method includes acquiring cardiovascular data from a subject, analyzing the cardiovascular data to determine a time trajectory for at least one cardiovascular parameter, and determining, using the time trajectory, a likelihood that the at least one cardiovascular parameter exceeds a threshold at one or more pre-determined time points. The method also includes determining a future cardiovascular state of the subject using the determined likelihood, and generating a report indicative of the future cardiovascular state of the subject. The method may also include estimating a dose response associated with at least one administered pharmaceutical agent for at least one cardiovascular parameter, and controlling the cardiovascular state of the subject at one or more pre-determined time points using the estimated dose response.

### CROSS-REFERENCE TO RELATED APPLICATIONS

This application is based on, claims priority to, and incorporates herein by reference in its entirety U.S. Provisional Application Ser. No. 62/035,177, filed Aug. 8, 2014, and entitled “METHODS FOR PREDICTION OF SUBJECT-SPECIFIC HEMODYNAMIC RESPONSE TO VASOPRESSORS.”

### BACKGROUND OF THE DISCLOSURE

The field of the invention relates to medical monitoring and intervention. More particularly, the invention relates to systems, device and methods for monitoring and controlling a cardiovascular (“CV”) state of a subject.

Critically-ill patients often require therapy using various pharmacological agents, in the form of acute or periodic treatment, in order to keep physiological parameters, such as blood pressure (“BP”), within a safe range. For instance, vasopressor infusion is typically performed to raise dangerously low BP, while treatment for hypertension can include infusion of nicardipine, or repeated discrete dosing using hydralazine. Selecting the proper pharmacological agent dose for optimized treatment often depends on the specific characteristics of the patient and as such requires careful assessment of the patient state. In many cases, there is a risk to insufficient medication, as well as a risk of excessive treatment.

Treatment is often made difficult by the fact that the patient's state can change over time, either as a result of a specific medical intervention or a change in the patient's condition. For example, the heart or vasculature of the patient may regain function or may become more unhealthy as time progresses, or a change in intravascular volume occurs, while a clinical intervention is being applied to the patient. As a consequence, the “ideal” dose for a pharmacological agent may also change with time. In addition, the effectiveness of pharmacological agents may wear off or diminish with time. Furthermore, a patient may also experience transient events affecting various the physiological parameters being monitored, such as BP. Such transient events are unlikely to persist, thus further complicating decisions on treatment timing and whether a treatment dose requires adjustment or not. Finally, clinicians may have multiple clinical obligations, and so minor dose adjustments that are unlikely to be clinically meaningful can be undesirable. As such, the ability to be selective about impactful dose-adjustments would be advantageous for a patient's clinical outcome.

As noted above, vasopressor medications are often infused to maintain BP in an optimal range. Vasopressors can act through one or more physiological mechanisms, including increasing resistance to blood exiting the arteries, which is commonly quantified as the total peripheral resistance (“TPR”), and vasopressor can increase cardiac output (“CO”) through increased heart rate (“HR”), cardiac contractility and decreased venous capacitance. The ultimate medical benefit of vasopressors is not increased BP per se, but increased blood flow to peripheral tissues driven by the increase in BP. In today's clinical practice, the infusion rate of vasopressor medications is adjusted by human clinicians. A complicating factor is that vasopressors can either improve blood flow to hypo-perfused peripheral tissues, via the increase in BP or, in some cases, can also decrease blood flow, via excessive increase in blood vessel resistance, depending on which effect is predominant. In addition, there is substantial individual variability in the physiological response to vasopressor therapy.

Standard clinical practice involves iteratively, and empirically, adjusting the infusion rate of vasopressors, seeking to maximize the expected beneficial effects relative to deleterious effects for the particulars of each patient. For instance, control of BP as an endpoint has been the focus of clinical practice guidelines for vasopressor use, where current recommendations dictate adjusting infusion rates to achieve a minimal mean arterial pressure (“MAP”) of at least 65 mmHg. In practice, clinicians empirically regulate vasopressor dose levels to achieve this target for MAP.

Although some analytical tools have been developed for informing the adjustment process of pharmaceutical agent dosages, they have not been widely adopted due to their inherent limitations. For example, existing technologies cannot predict patient-specific cardiac and vascular responses only based on non-invasive BP (“NIBP”) measurements. Also, existing technologies cannot provide patient-specific dose responses from limited hemodynamic information, but rather require a multitude of parameters and multiple observations. In addition, previous approaches for determining the required dose level often included analysis of the direct relationship between the administered pharmaceutical agent and the targeted parameter, such as the MAP, without taking into consideration the underlying physiology. This makes it difficult to predict the interactions as a result of multiple drug infusions and patho-physiological responses.

In light of the above, there is a need for improved systems and methods to accurately monitor and control CV conditions of a subject.

### SUMMARY OF THE DISCLOSURE

The present disclosure overcomes the aforementioned drawbacks by providing systems and methods for monitoring and controlling a cardiovascular state of a subject. Specifically, a novel approach for determining a current and/or future cardiovascular state of the subject is described, utilizing minimal and basic observations from an individual subject, such as blood pressure and heart rate. Determinations of current and future cardiovascular states may then be utilized to inform the administration of one or more pharmaceutical agent.

In some aspects of the disclosure, changes in blood pressure may be utilized to infer changes in the underlying cardiovascular state of the subject, and then estimate dose response relationships for the underlying cardinal cardiovascular parameters. In this manner, blood pressure as a function of an administered pharmaceutical agent, such as a vasopressor, may be predicted based on estimated cardiovascular state by extrapolating the dose response relationship.

In one aspect of the present disclosure, a system for monitoring a cardiovascular state of a subject is provided. The system includes at least one sensor configured to acquire cardiovascular data from a subject, and at least one processor configured to analyze the cardiovascular data to determine a time trajectory for at least one cardiovascular parameter, and determine, using the time trajectory, a likelihood that the at least one cardiovascular parameter exceeds a threshold at one or more pre-determined time points. The at least one processor is also configured to determine a future cardiovascular state of the subject using the determined likelihood, and generate a report indicative of the future cardiovascular state of the subject. The system also includes an output for displaying the report to a user.

In another aspect of the present disclosure, a system for controlling a cardiovascular state of a subject is provided. The system includes at least one sensor configured to acquire cardiovascular data from a subject, and at least one processor configured to receive the acquired cardiovascular data, and generate a cardiovascular model describing the cardiovascular state of the subject. The at least one processor is also configured to estimate a dose response associated with at least one administered pharmaceutical agent for at least one cardiovascular parameter defining the cardiovascular state, and control the cardiovascular state of the subject at one or more pre-determined time points using the estimated dose response.

In yet another aspect of the disclosure, a method for monitoring a cardiovascular state of a subject is provided. The method includes acquiring cardiovascular data from a subject using at least one sensor, and analyzing the cardiovascular data to determine a time trajectory for at least one cardiovascular parameter. The method also includes determining, using the time trajectory, a likelihood that the at least one cardiovascular parameter exceeds a threshold at one or more pre-determined time points, and determining a future cardiovascular state of the subject using the determined likelihood. The method further includes generating a report indicative of the future cardiovascular state of the subject.

In yet another aspect of the disclosure, a method for controlling a cardiovascular state of a subject is provided. The method includes receiving cardiovascular data acquired from a subject, and generating a cardiovascular model describing the cardiovascular state of the subject. The method also includes estimating a dose response associated with at least one administered pharmaceutical agent for at least one cardiovascular parameter defining the cardiovascular state, and controlling the cardiovascular state of the subject at one or more pre-determined time points using the estimated dose response.

The foregoing and other aspects and advantages of the invention will appear from the following description. In the description, reference is made to the accompanying drawings that form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

FIG. 1 is a schematic diagram of an example monitoring and control system in accordance with aspects of the present disclosure.

FIG. 2 is a flowchart setting forth steps of a process in accordance with aspects of the present disclosure.

FIG. 3 is a schematic diagram describing a cardiovascular model in accordance with aspects of the present disclosure.

FIG. 4 is a graphical illustration showing hemodynamic dose responses for different subject groups receiving epinephrine.

FIG. 5 is a schematic diagram illustrating training of phenomenological dose response relationships using vasopressor dose and blood pressure data.

FIG. 6 is a schematic diagram illustrating a cardiovascular model implementing phenomenological relationships between dose and cardinal cardiovascular parameters.

FIG. 7 is a graphical illustration comparing measured hemodynamic responses versus responses predicted in accordance with the present disclosure for different subject groups.

FIG. 8 are Bland-Altman plots associated with measured and predicted cardiovascular parameters, in accordance with the present disclosure.

FIG. 9 is a schematic diagram describing a cardiovascular model in accordance with aspects of the present disclosure.

FIG. 10 is a graphical illustration showing example hemodynamic responses to epinephrine averaged over several animal subjects.

FIG. 11 is another graphical illustration showing example hemodynamic responses to epinephrine obtained from an animal subject.

FIG. 12 is yet another graphical illustration showing intravenous versus hypothetical doses of epinephrine.

FIG. 13 is yet another graphical illustration comparing measured versus model-estimated cardiovascular parameters.

FIG. 14 is yet another graphical illustration showing model-estimated trends for different cardiovascular parameters.

FIG. 15 is yet another graphical illustration showing an estimated trend of cardiac output.

FIG. 16 is yet another graphical comparing measured versus predicted hemodynamic response for an animal subject.

FIG. 17 is a graphical illustration showing a correlation between measured and predicted blood pressure values.

FIG. 18 is another flowchart setting forth steps of a process in accordance with aspects of the present disclosure.

FIG. 19 is a graphical illustration showing an example of a determined time trajectory for blood pressure.

FIG. 20 is a graphical illustration showing a correlation between measured arterial pressure and determined probability for hypotension, in accordance with aspects of the present disclosure.

FIG. 21 is another graphical illustration showing a correlation between measured arterial pressure and determined probability for hypotension.

### DETAILED DESCRIPTION

The present disclosure describes a novel approach for monitoring and/or controlling a cardiovascular (“CV”) state of a subject using one or more administered pharmaceutical agents. In some aspects, the provided systems and methods are directed to monitoring and determining a present and potential future cardiovascular (“CV”) state of a subject in order to inform administration of at least one pharmaceutical agent, such as a vasopressor, by a clinician or a system suitable to do so. For instance, a time trajectory for at least one CV parameter, such as blood pressure (“BP”), may be determined to estimate a likelihood for exceeding a threshold at one or more pre-determined time points. As will be appreciated from descriptions below, this allows determination for the necessity, and/or timing of pharmaceutical agent administration, as well as possible benefit from a dose adjustment. This approach can result in minimized effort and fewer interruptions in clinical settings, where treatment dose is typically adjusted by a human clinician.

In some implementations, the provided systems and methods utilize a novel approach for selecting or optimizing the administration of one or more pharmaceutical agents. In particular, a CV state, or a change thereof, for a subject can be determined using various CV measurements. In some aspects, CV parameters describing the CV state of the subject, such as parameters related to cardiac output (“CO”) and total peripheral resistance (“TPR”), may be estimated or inferred, using a CV model, from basic vital sign measurements, such as heart rate (“HR”) and BP. As such, measurements or estimates associated with changes in the CV state of the subject may then be utilized to determine dose response relationships. Of note is that unlike previous methods attempting to directly analyze relationships between a drug and specific clinical endpoints, such as a particular BP, for example, ignoring underlying physiology, the present approach relies on estimates of the CV state of the subject to determine dose response relationships for controlling the condition of the patient.

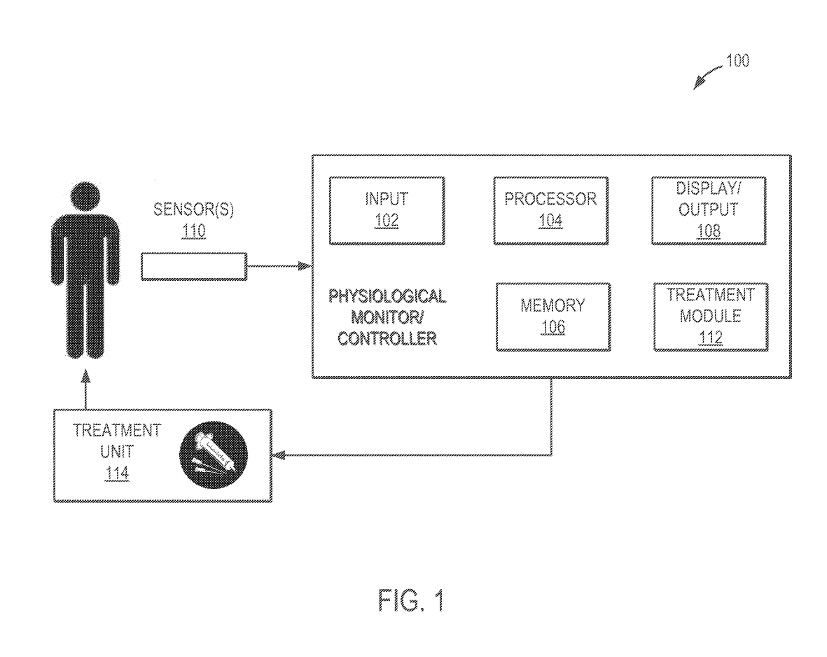

Referring particularly to FIG. 1, an example system 100 for monitoring and/or controlling a CV state of a subject is shown. In general, the system 100 may be any device, apparatus or system configured for carrying out instructions in accordance with the present disclosure. In some implementations, the system 100 may include an input 102, a processor 104, a memory 106, an output 108, and a number of sensors 110 configured to acquire HR, BP, and other physiological signals from a subject, intermittently or continuously. In some aspects, the sensors 110 may be configured to acquire data non-invasively from the subject. For example, the sensors 110 may include attachable or wearable sensors, such as oscillometric cuffs. In other aspects, the sensors 110 may be configured to acquire data from within a subject's anatomy, via insertable catheters or other interventional tools or devices. As shown in FIG. 1, the system 100 may optionally include a treatment module 112 for controlling a treatment unit 114 configured to deliver one or more pharmaceutical agents to the subject.

System 100 may operate independently or as part of, or in collaboration with, a computer, system, device, machine, mainframe, or server. In some aspects, the system 100 may be portable, such as a mobile device, smartphone, tablet, laptop, or other portable device, apparatus or personal monitoring system. In this regard, the system 100 may be any system that is designed to integrate with a variety of software and hardware capabilities and functionalities, in accordance with the present disclosure, and may be capable of operating autonomously and/or with instructions from a user or other system or device.

In particular, the input 102 may be configured to receive a variety of information from a user, a server, a database, and so forth, via a wired or wireless connection. The input 102 may include any number of input elements, for example, in the form of touch screens, buttons, a keyboard, a mouse, and the like, as well as compact discs, flash-drives or other computer-readable media. In some aspects, information provided via input 102 may include information associated with the subject, such as subject characteristic, including age, weight, medical condition, and so forth, as well as treatment type and duration, including selected pharmaceutical agent(s), dosing, and so forth. In some implementations, information associated with a population may also be provided via input 102. In addition, a user may provide selections for time range of data analysis, or one or more desirable thresholds for particular CV parameters. In some aspects, reference data associated with previous measurements obtained from the subject or population may also be provided via input 102, or alternatively retrieved from the memory 106 or other storage location.

In addition to being configured to carry out steps for operating the system 100 using instructions stored in the memory 106, the processor 104 may also be configured to monitor the CV state of a subject by receiving and processing CV data, and other data or information obtained using sensors 110 or input 102, or both, either continuously or intermittently. In some aspects, the processor 104 is configured to determine a time trajectory for one or more CV parameters and apply a statistical model, using the time trajectory, dosing and other information, to determine a likelihood that at least one CV parameter, such as BP, exceeds a threshold at one or more pre-determined time points. Specifically, the processor 104 may analyze CV data, acquired at various time points or over a time range, to determine a present and/or future CV state of the subject using the determined likelihoods. For instance, the processor 104 may determine the likelihood that a BP is higher, or lower, than a predetermined value for a certain duration over a selected analysis period.

The processor 104 may also determine a confidence interval (“CI”) for the determined time trajectory of the CV parameter(s), for one or more points in time. This may include considering information from prior analyses, such as information obtained from data obtained from a population, and/or previous observations from the subject. Also, in conducting a statistical analysis, the processor 104 may utilize a number of statistical techniques including Monte Carlo simulations, regression model analyses, and other formulae that provide a statistical result based on predicted time trajectories, confidence intervals, dose, and other important clinical factors.

The processor 104 may also be configured to carry out steps for controlling the CV state of a subject at one or more pre-determined time points. In some aspects, the processor 104 may take into consideration computed likelihoods, as described above. Using received or acquired CV data, as will be described, the processor 104 may also be configured to generate a CV model describing the CV state of the subject, and estimate a dose response for at least one administered pharmaceutical agent using CV parameters defining the CV state. In some aspects, some CV parameters, such as parameters related to CO and TPR, may be estimated or inferred by the processor 104 using basic vital sign measurements, such as HR, mean arterial pressure (“MAP”), systolic BP (“SBP”), and diastolic BP (“DBP”). As will be described, the processor 104 may then determine dose response relationships for one or more pharmaceutical agents using as few as two parameter measurements or estimates reflecting changes in the CV state of the subject with dose. Such dose response relationships may then be used inform a clinician regarding potential hemodynamic responses to pharmaceutical agents, such as vasopressors, to be administered to the subject.

In some implementations, the processor 104 may be configured to combine various types of information, such as a determined time trajectory for one or more CV parameters, confidence intervals, user selections, dose response relationships and other information, to determine an optimized treatment for controlling the CV state of the subject in accordance with clinical requirements. The processor 104 may then communicate with a treatment module 112 to control the treatment unit 114 for delivering the optimized treatment to a subject. As described, the condition of a subject may vary with time, and hence the processor 104 may be configured to determine an optimized treatment iteratively or periodically to account for changing clinical conditions. For example, the processor 104 may adapt or modify an infusion rate or dose of one or more pharmaceutical agents.

The processor 104 may also be configured to generate a report provided to a user or clinician via an output 108, in the form of an audio and visual display. The report may include a variety of information and data associated with the subject. For instance, the report may include an indication regarding a present or future CV state of the subject. In some aspects, the report may display a time trajectory for one or more CV parameters along with respective confidence intervals, and provide an alert or notification related to a likelihood that one or more thresholds are exceeded at one or more pre-determined time points. In addition, the report may provide an indication to a clinician regarding the timing and dosing of one or more pharmaceutical agents for maintaining or achieving a target CV state.

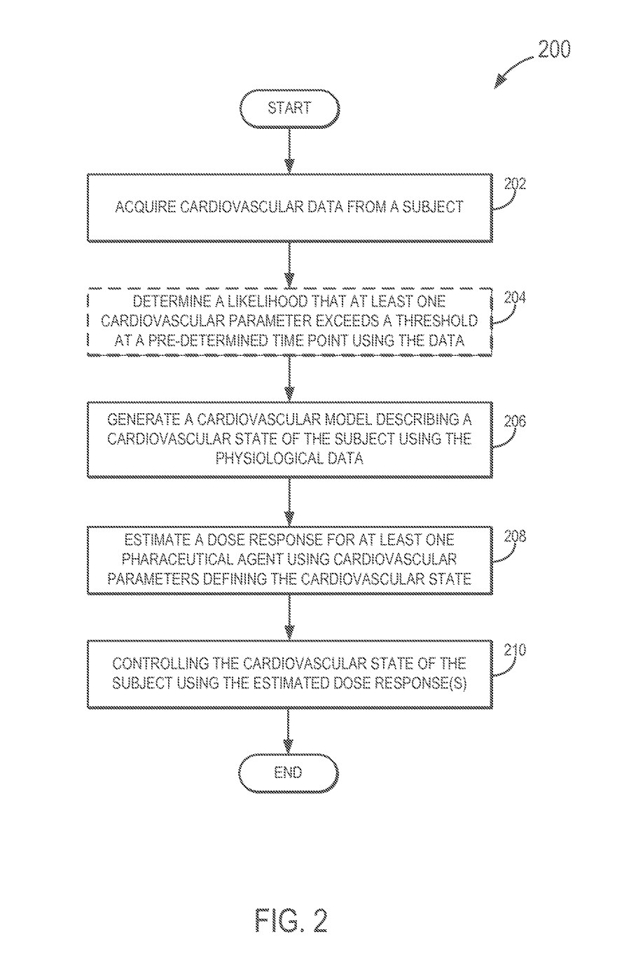

Turning to FIG. 2, steps of a process 200 for controlling a CV state of a subject are shown, which may be carried out for example, using a system as described with reference to FIG. 1. The process 200 may begin at process block 202 where CV data, and other physiological data, may be acquired from a subject. In some aspects, a likelihood that at least one CV parameter exceeds a selectable threshold or parameter range at a pre-determined time points may be optionally determined, as indicated by process block 204. In addition, a determination can be made whether at least one CV parameter defining the cardiovascular state of the subject exceeds one or more thresholds for a selected duration. This would allow the identification of brief or transient events which might not require direct intervention. In this manner, the number of actions or notifications for controlling the state of the subject may be minimized or restricted to those conditions when, statistically-speaking, specific CV parameters are likely to persist outside pre-determined or safe values for a certain amount of time, for instance.

At process block 206, a CV model describing a CV state of the subject is generated using the acquired CV data. Parameters defining the CV state can then be utilized to estimate a dose response for at least one pharmaceutical agent, as indicated by process block 208 and described below. Such dose responses can then be utilized to control the CV state of a subject, as indicated by process block 210, either by a clinician or a system configured to do so. In some aspects, a determination can be made at process block 210 to identify whether action needs to be taken to control the cardiovascular state of the subject, in accordance with a dose adjustment computed using the estimated dose response. For example, it may advantageous to identify whether a computed dose adjustment exceeds a pre-determined threshold, to ensure that meaningful dose adjustments are performed. In addition, information associated with prior treatment, or prior dose adjustment may also be helpful in making such determination. For example, it would be advantageous to receive information about a prior dose adjustment in order to allow sufficient time for the medication to take effect before making further adjustments. This may result in fewer number of clinical interventions or interruptions, particularly when clinicians are involved in the processes for controlling patient.

Although descriptions provided herein are directed to prediction of hemodynamic responses of vasopressors, it may be appreciated that the approach described may be readily extended to a variety of pharmaceutical agents and conditions. By way of non-limiting examples, pharmaceutical agents for use in accordance with the present disclosure can include medications for increasing blood pressure and/or heart rate, such as Epinephrine, Noradrenaline, Phenylephrine, Dobutamine, Dopamine, Ephedrine, Midodrine, Digoxin, Amrinone, Milrinone, Isoproterenol, Vasopressin, and others, as well as medications for lowering blood pressure and/or heart rate, such as Diltiazem, Verapamil (and other calcium channel blockers), Clonidine, Hydralazine, Nitroprusside, Nitroglycerine, Esmolol, Nifedipine, Nicardipine, Labetolol (and other alpha and beta mixed blockers), Esmolol (and other beta-blockers), Clonidine, as well as other pharmaceutical agents known for treating hypotensive or hypertensive conditions.

Clinicians typically titrate the vasopressor drug levels based on BP, even though changes in BP actually reflect changes in CO and TPR, the underlying cardinal CV parameters. The present disclosure recognizes that relationships between vasopressor dose level and cardinal parameters are more consistent than those between vasopressor dose and BP. As such, the present approach is directed to estimating dose response relationships for underlying cardinal parameters. In some aspects, CO and TPR are not directly measured, then the effects of the vasopressor agent on the cardinal parameters can be estimated or inferred. Herein, a specific algorithm that uses non-continuous measurements of SBP, MAP, DBP, and HR are used to infer cardinal parameters related to CO and TPR. For example, HR and BP measurements may be obtained using non-invasive oscillometric cuffs, which are typical of the sparse vital signs available during the early stabilization (i.e., resuscitation) of patients with circulatory shock, before continuous BP data (via indwelling arterial catheterization) or direct CO measurements are available.

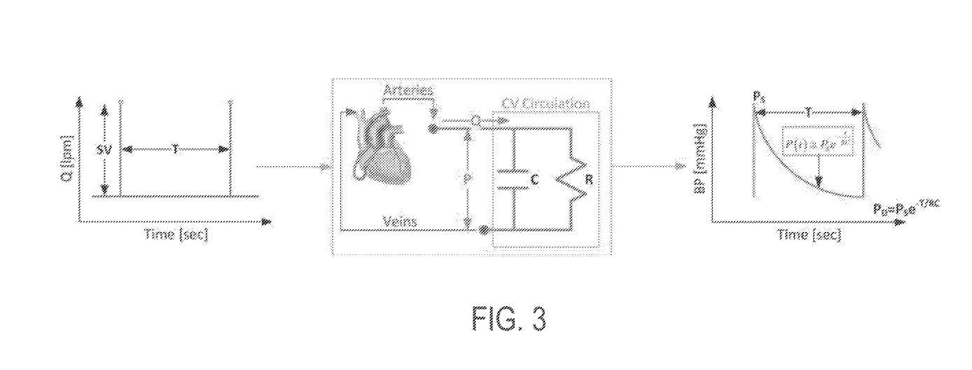

As such, given sparse data, a simple CV model, such as the WK model, may be adequate. In such approach, the aortic flow may be approximated as a train of impulses, and a resistor and capacitor may be utilized to represent TPR and arterial compliance (“AC”), respectively, as shown in FIG. 3. This may be mathematically expressed as follows:

$$\frac{dP ( t )}{dt} = - \frac{1}{RC} P ( t ) + \frac{1}{C} δ V \cdot δ ( t ) \qquad ( 1 )$$

where 0≦t≦T, P(t) represents BP, R, C and δV represent TPR, AC, and stroke volume (“SV”), respectively, and T is the heart period. In Eqn. 1, δ(t) is the direct Delta function. Using the SV index (“SVI”), Eqn. 1 becomes

$$\frac{dP ( t )}{dt} = - \frac{1}{\overline{RC}} P ( t ) + \frac{1}{\overline{C}} δ \overline{V} \cdot δ ( t ) \qquad ( 2 )$$

where δV̅ is the SVI, R̅ (in mmHg·min·m²/l) and C̅ (in ml/mmHg/m²) are defined as the TPR index (“TPRI”), computed by dividing MAP by SVI and HR, and AC index (“ACI”), respectively. Solving Eqn. 2 for BP using a convolution integral yields

$$P ( t ) = {P}_{d} {e}^{- \frac{t}{\overline{RC}}} + {\int }_{τ = 0}^{t} {e}^{- \frac{t}{\overline{RC}} ( t - τ )} \frac{1}{\overline{C}} δ \overline{V} \cdot δ ( τ ) d τ = {P}_{d} {e}^{- \frac{t}{\overline{RC}}} + \frac{δ \overline{V}}{\overline{C}} {e}^{- \frac{t}{\overline{RC}}} \qquad ( 3 )$$

which is valid for each heart period, as in FIG. 3. From Eqn. 3, the following relationships between BP, TPRI and SVI, scaled by ACI, may be obtained:

$$\begin{matrix}{P}_{m} = \overline{R} \cdot HR \cdot δ \overline{V} = \overline{RC} \cdot HR \frac{δ \overline{V}}{\overline{C}} & ( 4 a ) \\ {P}_{s} = {( 1 - {e}^{- \frac{T}{\overline{RC}}} )}^{- 1} \frac{δ \overline{V}}{\overline{C}} & ( 4 b ) \\ {P}_{p} = {P}_{s} - {P}_{d} = \frac{δ \overline{V}}{\overline{C}} & ( 4 c )\end{matrix}$$

where Pₛ, Pₘ, P_{d} are SBP, MAP and DBP, respectively, and Pₚ is the pulse pressure (“PP”). Given measurements of SBP, MAP and PP, as well as HR for a given vasopressor dose level, the corresponding values of R̅C̅ and

$$\frac{δ \overline{V}}{\overline{C}}$$

can be determined using Eqns. 4. For this purpose, the following multi-objective optimization problem may be formulated based on the relationship between BP versus R̅C̅ and

$$\frac{δ \overline{V}}{\overline{C}} :$$

$$\{ {\overline{RC}}^{*} , \frac{δ {\overline{V}}^{*}}{\overline{C}} \} = arg min J ( \overline{RC} , \frac{δ \overline{V}}{\overline{C}} ) = arg min \{ {‖ [ \begin{matrix}{F}_{1} ( \overline{RC} , \frac{δ \overline{V}}{\overline{C}} ) \\ {F}_{2} ( \overline{RC} , \frac{δ \overline{V}}{\overline{C}} ) \\ {F}_{3} \frac{δ \overline{V}}{\overline{C}}\end{matrix} ] ‖}_{∞} \} \qquad ( 5 )$$

where R̅C̅* and

$$\frac{δ {\overline{V}}^{*}}{\overline{C}}$$

are the optimal scaled TPRI and SVI associated with a particular vasopressor dose, and Fᵢ, i=1, 2, 3 are specified as follows:

$$\begin{matrix}{F}_{1} ( \overline{RC} , \frac{δ \overline{V}}{\overline{C}} ) = [ {P}_{m} - \overline{RC} \cdot HR \cdot \frac{S \overline{V}}{\overline{C}} ] / {P}_{m} & ( 6 a ) \\ {F}_{2} ( \overline{RC} , \frac{δ \overline{V}}{\overline{C}} ) = [ {P}_{s} - {( 1 - {e}^{- \frac{T}{RC}} )}^{- 1} \frac{S \overline{V}}{\overline{C}} ] / {P}_{s} & ( 6 b ) \\ {F}_{3} ( \frac{δ \overline{V}}{\overline{C}} ) = [ {P}_{p} - \frac{S \overline{V}}{\overline{C}} ] / {P}_{p} & ( 6 c )\end{matrix}$$

which are derived from Eqns. 4. From

$${\{ d , HR , {P}_{{s}^{′}} {P}_{{m}^{′}} {P}_{d} \}}_{i} , i = 1 , … N {\{ {\overline{RC}}^{*} , \frac{δ {\overline{V}}^{*}}{\overline{C}} \}}_{i}$$

can be obtained, where {•}ᵢ denotes dose (“d”) and CV responses (HR and BP as well as scaled TPRI and SVI) corresponding to the i-th dose, while N is the number of dose levels at which BP measurements are taken to train the model. Using the approach described above, a subject's complete CV state may be estimated.

To estimate dose responses, as described with regard to FIG. 2, a set of phenomenological models may be estimated between a pharmaceutical dose level, such as a vasopressor, and the cardinal CV parameters. Advantageously, this approach permits the estimation of individualized dose responses using observations from as few as two dose levels. In particular, because different vasopressors affect the cardinal CV parameters differently, specific phenomenological models may be used to reproduce phenomena that are vasopressor-dependent. For instance, a different specific model might be employed for epinephrine as opposed to norepinephrine. In addition, as discussed below, it may also be appropriate to modify the methodology for low dose epinephrine (where beta effects are more significant than alpha effects) versus high-dose epinephrine (where alpha effects are more significant than beta effects).

By way of example, the above-described phenomenological approach was applied to a dataset including hemodynamic dose response relationships for epinephrine. The dataset included hemodynamic responses of 14 normotensive young (“NY”; 30+/−2 yr) and 18 normotensive old (“NO”; 60+/−2 yr) subjects as well as 10 hypertensive young (“HY”; 36+/−1 yr) and 17 hypertensive old (“HO”; 59+/−1 yr) subjects. As per the original report, normotensive and hypertensive BP was defined as having values less than 130 mmHg SBP/85 mmHg DBP and greater than 140 mmHg SBP/95 mmHg DBP, respectively. The dataset provided BP data as a function of epinephrine dose (SBP, MAP and DBP), measured using an oscillometric arm BP cuff. The dataset also included measurements of HR and SVI, measured using echocardiography, wherein the SVI data were used to provide the gold-standard measurements against which the accuracy of the prediction of the present approach was evaluated. In the original experimental protocol, following a rest period of at least 60 min, epinephrine was administered in consecutive 8 min intervals, at 20 ng/kg/min, 40 ng/kg/min, 80 ng/kg/min, 120 ng/kg/min and 160 ng/kg/min. Typically, the beta effects of epinephrine are dominant when the dose is less than about 50 ng/kg/min, while its alpha effects do not dominate until doses of about 100 ng/kg/min. In this dataset, hemodynamic measurements were performed at steady state before epinephrine administration and then during the last 2-3 min of each consecutive epinephrine dosing interval.

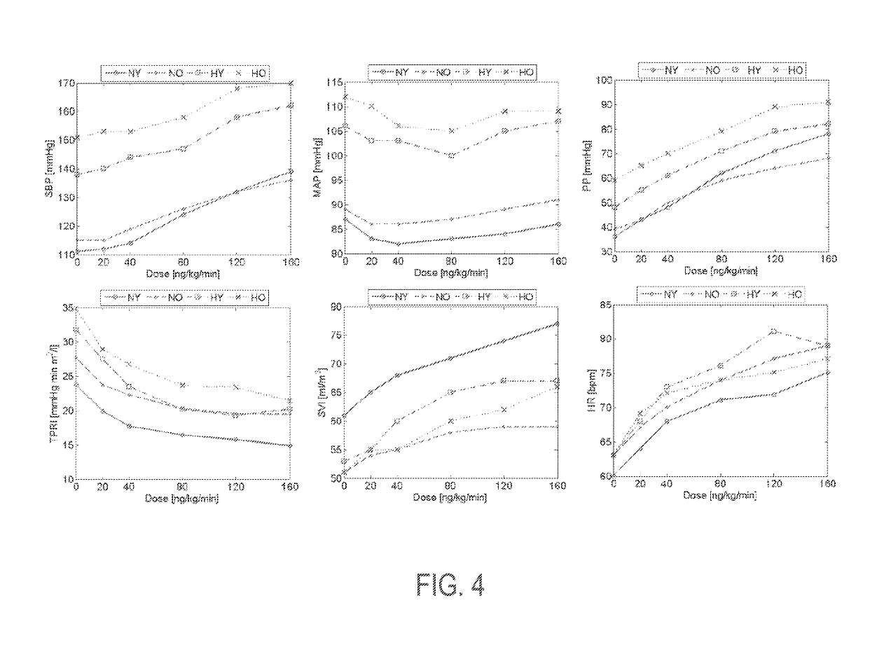

FIG. 4 shows the pooled hemodynamic responses from the four sets of subjects to different epinephrine doses, including SBP, MAP and PP as well as TPRI, SVI and HR. Their ranges are summarized in Table 1. Note that inter-individual variability was not considered in this study.

TABLE 1
Physiologic range of epinephrine dose-
dependent hemodynamic responses.

| | | | | | | SVI |
|---|---|---|---|---|---|---|
| | | HR | SBP | MAP | DBP | [ml/ |
| | | [bpm] | [mmHg] | [mmHg] | [mmHg] | m²] |
| Baseline Value | | 60-63 | 111-151 | 87-112 | 75-92 | 51-61 |
| Hemo- | 020 ng/ | 64-69 | 112-153 | 83-110 | 69-88 | 54-65 |
| dynamic | kg/min | | | | | |
| Responses | | | | | | |
| | 040 ng/ | 68-73 | 114-153 | 82-106 | 66-83 | 55-68 |
| | kg/min | | | | | |
| | 080 ng/ | 71-76 | 124-158 | 83-105 | 62-79 | 58-71 |
| | kg/min | | | | | |
| | 120 ng/ | 72-81 | 132-168 | 84-109 | 61-79 | 59-74 |
| | kg/min | | | | | |
| | 160 ng/ | 75-79 | 136-170 | 86-109 | 61-80 | 59-77 |
| | kg/min | | | | | |

The challenge of estimating an individualized dose response relationship with only two observations resides the complexity of effects caused by pharmaceutical agents like epinephrine. Lower doses of epinephrine (e.g. <50 ng/kg/min) decrease TPR over its baseline value (beta agonist effect), whereas higher doses (e.g., >100 ng/kg/min) activate alpha receptors and increase TPR. Values for HR and inotropy (affecting SV) are primarily increased by beta agonist action. To reproduce the anticipated responses of HR, TPRI and SVI accurately while minimizing the amount of a priori data required to train the models, the following phenomenological models dictating the dose dependence of HR, R̅C̅ and

$$\frac{δ \overline{V}}{\overline{C}}$$

were developed:

$$\begin{matrix}HR = {f}_{1} ( d , {θ}_{1} ) = {( {k}_{1 H} d + {k}_{2 H} )}^{0.1} , {θ}_{1} = \{ {k}_{1 H} , {k}_{2 H} \} & ( 7 a ) \\ \overline{RC} = {f}_{2} ( d , {θ}_{2} ) = \frac{1}{{k}_{1 R} d + {k}_{2 R}} + [ {k}_{3 R} d + {k}_{4 R} ] σ ( d , {d}_{0} ) , \\ {θ}_{2} = \{ {k}_{1 R} , {k}_{2 R} \} & ( 7 b ) \\ \frac{δ \overline{V}}{\overline{C}} = {f}_{3} ( d , {θ}_{3} ) = {k}_{1 V} d + {k}_{2 V} - [ {k}_{3 V} d + {k}_{4 V} ] σ ( d , {d}_{0} ) , \\ {θ}_{3} = \{ {k}_{1 V} , {k}_{2 V} \} & ( 7 c )\end{matrix}$$

where d is the drug dose level, k_{1H}, k_{2H}, k_{1R}, k_{2R}, k_{1V}, k_{2V} are empirical constants that quantify the beta agonist effect, whereas k_{3R}, k_{4R}, k_{3V}, k_{4V} are constants dictating the alpha agonist effects. The function σ(d, d₀) in Eqns. 7b and 7c is intended to activate the alpha agonist action in the high dose region and is defined as follows:

$$σ ( d , {d}_{0} ) = \{ \begin{matrix}0 , & d < {d}_{0} \\ 1 , & d \geq {d}_{0}\end{matrix} . \qquad ( 7 d )$$

These phenomenological models are able to capture dose-dependent behavior of the cardinal CV parameters. That is, the phenomenological models can capture an increasing HR with increased d, which tapers off at higher dose levels, an increasing SVI with increasing d, which also tapers off at higher levels, as well as a decreasing TPRI with increasing d due to beta agonist effects, until alpha agonist effects become evident.

In accordance with some aspects, as few as two observations could be utilized. As such, even the simplified dose response models described above include too many unknowns. Accordingly, the model parameters driving the most inter-subject variability can be solved while population-based values may be utilized for the others, in order to reduce the number of unknowns. For instance, examining FIG. 4 it is apparent that alpha agonist effects did not drive the subject's CV responses, and given this dosing regime (less than 160 ng/kg/min) most of the inter-subject CV variability was driven by the beta agonist effects (namely k_{1H}, k_{2H}, k_{1R}, k_{2R}, k_{1V}, k_{2V}). As such, the parameters associated with beta agonist effects can be estimated, while relying on population-averaged values for the alpha effects, namely k_{3R}, k_{4R}, k_{3V}, k_{4V} and d₀. As such, the phenomenological models of Eqns. 7 would then have two unknowns, which can be solved using data from two observations.

For example, the beta agonist component of the phenomenological models of Eqns. 7b and 7c may be modeled using the epinephrine dose and BP response data by first calculating R̅C̅* and

$$\frac{δ {\overline{V}}^{*}}{\overline{C}}$$

corresponding to 0 ng/kg/min and 20 ng/kg/min. As such,

$$\overline{RC} = \frac{1}{{k}_{1 R} d + {k}_{2 R}} and \frac{δ \overline{V}}{\overline{C}} = {k}_{1 V} d + {k}_{2 V}$$

may then be fitted to R̅C̅* and

$$\frac{δ {\overline{V}}^{*}}{\overline{C}}$$

at the two dose levels using Eqns. 8b and 8c, respectively. In addition, R̅C̅ and

$$\frac{δ \overline{V}}{\overline{C}}$$

associated with high epinephrine dose levels, namely 80, 120 and 160 ng/kg/min) may then be predicted using the beta agonist model thus obtained. Also, R̅C̅* and

$$\frac{δ {\overline{V}}^{*}}{\overline{C}}$$

may be directly calculated using Eqns. 6 for high epinephrine dose levels using experimental data. The discrepancy between direct versus model-predicted R̅C̅ and

$$\frac{δ \overline{V}}{\overline{C}}$$

may also be calculated for all subject groups, regarded as the contribution from the alpha agonist action. Furthermore, the alpha agonist components of Eqns. 7b and 7c, namely k_{3R}, k_{4R}, k_{3V}, and k_{4V} may then be optimized to minimize the discrepancy in R̅C̅ and

$$\frac{δ \overline{V}}{\overline{C}} .$$

Once the alpha agonist parameters are determined as described above, the phenomenological model may then be trained by first fixing the optimal population values in Eqns. 7b and 7c. Then, the optimization Eqn. 5 may be solved to obtain R̅C̅* and

$$\frac{δ {\overline{V}}^{*}}{\overline{C}}$$

for each dose level, such as a baseline and one dose value. Values for R̅C̅* and

$$\frac{δ {\overline{V}}^{*}}{\overline{C}} ,$$

together with HR measurements, may then be used to train the phenomenological models of Eqns. 7 via Eqns. 8. The phenomenological dose response models may then be used to predict hemodynamic responses for various CV parameters including SBP, MAP and DPB as well as the trends of TPR, SVI, and COI.

Quantitatively, the phenomenological models of Eqns. 7 dictating the reliance of HR, R̅C̅ and

$$\frac{δ \overline{V}}{\overline{C}}$$

on vasopressor dose level can be fitted to the acquired data. For instance, given pairs of data,

$${\{ d , HR , {\overline{RC}}^{*} , \frac{δ {\overline{V}}^{*}}{\overline{C}} \}}_{i} , i = 1 , … N ,$$

the optimal parameters θ₁, η₂ and θ₃ may be determined using a least-squares optimization process as follows:

$$\begin{matrix}{θ}_{1}^{*} = arg {min}_{{θ}_{1}} {\sum }_{i = 1}^{N} {[ HR - {f}_{1} ( d , {θ}_{1} ) ]}_{i}^{2} & ( 8 a ) \\ {θ}_{2}^{*} = arg {min}_{{θ}_{2}} {\sum }_{i = 1}^{N} {[ {\overline{RC}}^{*} - {f}_{2} ( d , {θ}_{2} ) ]}_{i}^{2} & ( 8 b ) \\ {θ}_{3}^{*} = arg {min}_{{θ}_{3}} {\sum }_{i = 1}^{N} {[ \frac{δ {\overline{V}}^{*}}{\overline{C}} - {f}_{1} ( d , {θ}_{3} ) ]}_{i}^{2} & ( 8 c )\end{matrix}$$

where [•]ᵢ is the expression evaluated using

$${\{ d , HR , {\overline{RC}}^{*} , \frac{δ {\overline{V}}^{*}}{\overline{C}} \}}_{i} .$$

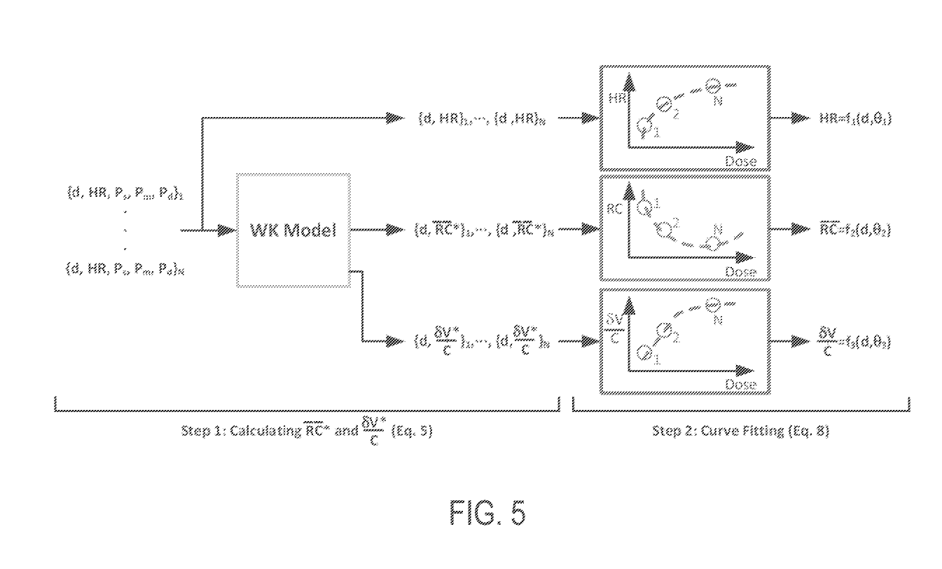

This process of training to an individuals' data is shown in FIG. 5, whereby basic vital signs, such as HR, SBP, MAP and DBP measured at two or more doses, are utilized to determine CV parameters defining a CV state of the subject.

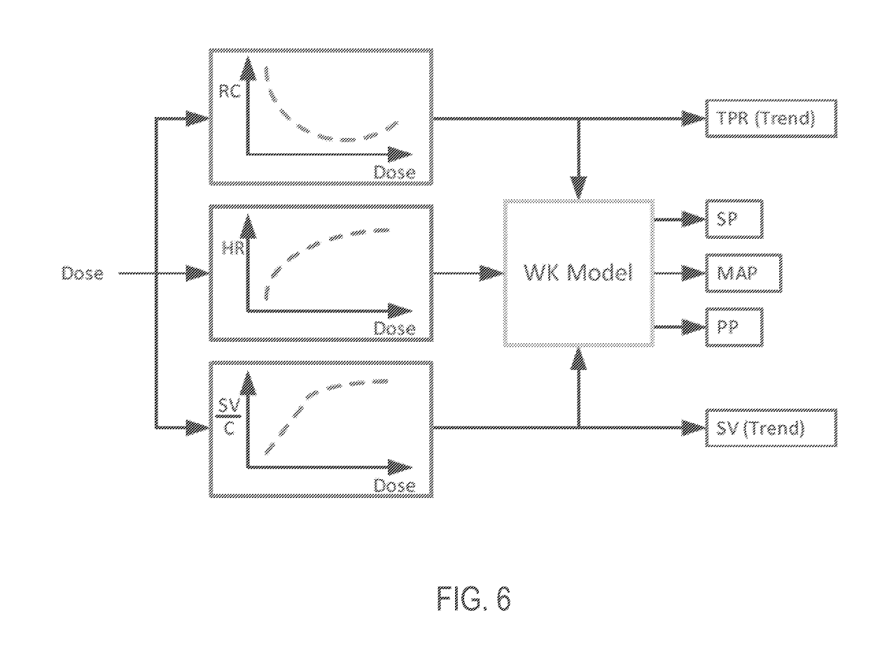

Using the WK model with the phenomenological dose response relationships, as described by Eqns. 7 individualized with θ*ᵢ, i=1, . . . 3 obtained using Eqns. 8, the hemodynamic responses for vasopressor dose levels can be predicted solely based on the vasopressor dose as follows (illustrated in FIG. 6). Values obtained for HR, R̅C̅ and

$$\frac{δ \overline{V}}{\overline{C}}$$

may be extrapolated using individualized phenomenological models in accordance with Eqns. 7 with the new vasopressor dose as input. Next, HR, R̅C̅ and

$$\frac{δ \overline{V}}{\overline{C}}$$

thus extrapolated may be substituted in Eqns. 4 to predict SBP, MAP and PP associated with the vasopressor dose. Further, assuming that the short-term variability of AC is small and it an essentially be regarded as constant over short time window, R̅C̅ and

$$\frac{δ \overline{V}}{\overline{C}}$$

predicted from the phenomenological models in Eqns. 7 can be regarded as predictions of TPRI and SVI (with unknown scale). Finally, the CO index (“COI”) can be predicted as the product of HR and

$$\frac{δ \overline{V}}{\overline{C}}$$

predicted with the phenomenological models of Eqns. 7.

By way of example, the above framework was used to demonstrate the prediction of dose responses to epinephrine infusion. Specifically, discrete BP measurements, namely SBP, MAP, DBP, and HR measures from only two different epinephrine dose levels were used to train the phenomenological dose response models of Eqns. 7. The measurements obtained at zero and a finite dose value may be utilized. The models were then used to predict cardinal CV parameters, namely TPRI, SVI and HR for dose levels not used in the training phase. This approach was applied to each set of subjects described with respect to FIG. 4, whereby predicted hemodynamic responses were compared with in-vivo experimental data, namely reported MAP and HR data, and RC and SVI/C estimated from reported BP data using Eqns. 6.

For each set of subjects, different ways of training were studied. In particular, to explore the performance for a patient requiring a vasopressor wean (i.e. prediction for a reduced dosage), BP and HR data from 0 ng/kg/min and 160 ng/kg/min dose values were used to predict TPRI, SVI, HR and BP responses to 20, 40, 80 and 120 ng/kg/min. To explore the performance for a patient in flux (i.e. prediction required for both increasing and decreasing doses), BP and HR data from 0 ng/kg/min and 80 ng/kg/min was used to predict TPRI, SVI, HR and BP responses to 20, 40, 120 and 160 ng/kg/min. In addition, to explore the performance for a patient receiving an inadequate vasopressor dose, BP and HR data using 0 ng/kg/min and 20 ng/kg/min was used to predict TPRI, SVI, HR and BP responses to 40, 80, 120 and 160 ng/kg/min. The approach was assessed in terms of prediction errors on SBP, MAP and PP, as well as the goodness of fits between measured versus model-predicted TPRI, SVI and COI in terms of the coefficient of determination (CoD; r² value) and the Bland-Altman statistics (i.e., the limits of agreement). A linear regression analysis was conducted on R̅C̅* and

$$\frac{δ {\overline{V}}^{*}}{\overline{C}}$$

to calibrate them against measured TPRI and SVI before quantifying the goodness of fits, thereby eliminating the effect of unknown AC.

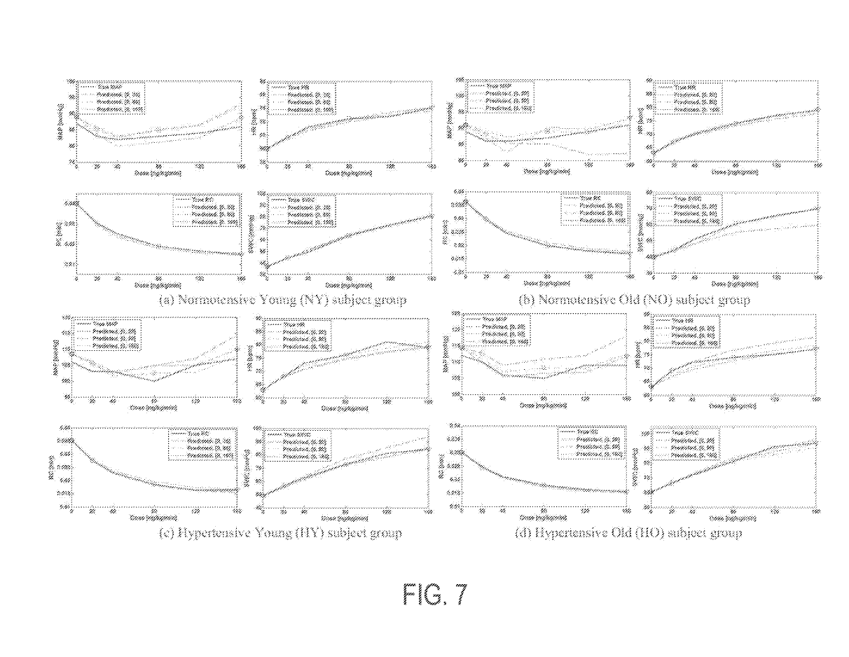

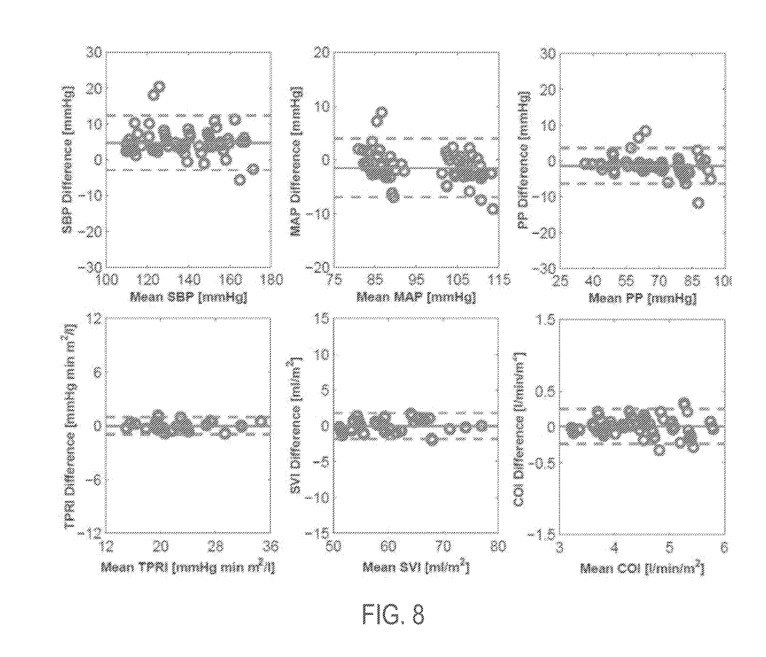

As appreciated from FIG. 7, showing predicted and measured hemodynamic responses for each group of subjects, the framework utilized was able to accurately predict absolute responses of BP and HR. In addition, the phenomenological dose response models could reproduce the biphasic behavior of MAP expected in response to epinephrine infusion, namely a decrease in MAP at lower dose levels and an increase at high dose levels. In terms of RMSE, the difference between the actual and model-predicted SBP, MAP and PP values, aggregated across the four sets of subjects, were less than 6% of the respective underlying values when trained with {0 ng/kg/min, 20 ng/kg/min}, less than 4% when trained with {0 ng/kg/min, 80 ng/kg/min}, and less than 4% when trained with {0 ng/kg/min, 160 ng/kg/min}, respectively. The ability to estimate cardinal CV parameters' responses was also compared to new doses, as shown in FIG. 7. The correlation between the actual and model-predicted cardinal CV parameters (TPRI, SVI, and COI as calculated by the product of SVI and HR) were high, namely the r² values aggregated across the four sets of subjects, were higher than 0.96 regardless of the training doses used. These results also support the strategy utilized wherein individualized beta agonist parameters were used, while fixing alpha agonist parameters to population-averaged values. Finally, the Bland-Altman analysis performed indicated that the model-predicted hemodynamic responses were in good agreement with their measured counterparts, as shown in FIG. 8. The biases were observed to be very small, indicated by the solid horizontal lines in FIG. 8. In addition, the limits of agreement were also tight, namely the confidence intervals associated with SBP, MAP and PP as well as TPRI, SVI and COI were consistently less than 8% of the respective underlying values (calculated as the average between actual versus model-predicted values).

As described, rather than directly obtaining a dose response relationship between BP and vasopressor dose, the complete CV state of the subject was determined using basic HR and BP measurements and unmeasured cardinal CV parameters inferred therefrom. This approach offers two potential advantages. First, the estimation of multiple CV parameters defining the CV state allows for more sophisticated dose adjustment. Second, this approach allows for more accurate predictions. For instance, relationships between vasopressor dose level and the unmeasured, cardinal parameters, such as SVI and TPRI, may in fact be more consistent than the relationship between vasopressor dose and the measured parameters, such as BP.

As appreciated from the above, the present framework can accurately predict how different individual patients may respond to various doses of administered pharmaceutical agents. For instance, consider how the present approach was able to extrapolate reliably beyond its training region. Specifically, given training data during low-to-medium dose (beta-receptor agonist) infusion, this approach was able to anticipate that medium-dose infusion would lead to further reduction in MAP (due to maximum beta agonist effect) whereas high-dose infusion would then increase MAP (since alpha agonist effect becomes dominant). This effect was observed consistently for each of the three studies involving the NY, HO, and HY groups, and two of the studies involving the NO group, with only in one of sixteen trials where the anticipated trend of MAP was not seen, namely in the isolated NO group study with training data from {0 ng/kg/min and 20 ng/kg/min} likely due to limited accuracy in SVI prediction for higher epinephrine doses. It must be emphasized that reproduction of this physiologically relevant dose-dependent MAP behavior was not trivial, since the present model was not fitted directly to the pair of epinephrine dose and MAP, and only two sets of observations were used to train the model. Specifically, MAP was inferred via the model-based relationship of Eqns. 4 from HR, TPRI and SVI, which were predicted from the phenomenological models of Eqns. 7. This demonstrates how the combination of the CV model and phenomenological dose response relationships for cardinal CV parameters can yield advantageous performance.

Analyzing a dataset consisting of non-continuous measurements of SBP, MAP, DBP, and HR, as described, the complete CV state was determined. Given such rudimentary measurements, the WK model was deemed sufficient to infer the underlying CV state in terms of the cardinal CV parameters related to CO and TPR. However, more advanced models and methods to infer the CV state may be possible given more profound and richer data, such as BP and flow waveforms, for example. In particular, given a BP waveform, the current WK model could be replaced by the long-time interval analysis or the improved WK-model-based method, to better estimate TPR and SV. For patients with a Swann-Ganz catheter, direct CO and TPR measurements could be used. In some populations, the WK model may not account for certain effects leading to poor performance. For instance, AC changes as a function of BP. In the presently analyzed dataset, the range of BP was small, and so, presumably, was the range of compliance. This may not be the case for patients with drastic BP changes, as might be observed in circulatory shock. In such cases, modifications of the methodology may need to be considered, including use of population-based model of pressure-dependent AC, and direct measurement of CO and TPR rather than model-based estimation, as is clinically available for some critically ill patients. In addition, measurements of a parameter correlated with AC, such as pulse-transit time, may also be utilized.

Presently, given that there is appreciable variability between how different patients respond to administered pharmaceutical agents, a novel approach for generating individualized dose response relationships was implemented. In some aspects, a minimal number of individualized observations may be utilized, with as few as two, which can be useful even during the earliest stages of critical care. To determine dose response relationships given just two observations, the number of unknown parameters utilized in the phenomenological models was reduced by relying on prior knowledge regarding the biggest drivers of inter-subject variability. As described, population-averaged values for the alpha agonist effects were utilized, because they were less dominant than their beta counterparts. The result was encouraging performance as indicated in FIGS. 7 and 8. In some aspects, this strategy could be modified, for instance, by modifying the phenomenological models utilized according to the effects of the different pharmaceutical agents as well as the specific dosing employed. For instance, the decision to solve for individual beta effects or alpha effects would likely be a function of the dosing regime. As such, for patients receiving high-dose epinephrine, for example, it is quite likely that inter-subject variability will be driven more by alpha effects, and hence it may be preferable to individualize the alpha effect models and employ population-based coefficients for the beta effects, or to require additional observations, which would allow for solving for additional unknowns. In some aspects, reference datasets may also be examined to evaluate how to employ any such a priori knowledge.

In particular, vasopressor-inotropes are medications used to increase arterial BP and CO in critically ill patients presenting with CV compromise/shock. Vasopressor-inotropes can operate through different physiological intermediate mechanisms, such as (i) increasing resistance to blood distributing to the body (TPR), (ii) increasing CO through enhanced cardiac contractility and HR, and (iii) reduced venous capacitance. Raising BP is not the only medical advantage behind the use of vasopressor-inotropes. In fact, increased blood flow and oxygen delivery to peripheral tissues can in some situations be more important outcomes that can be attained by the raised BP and cardiac function.

Nowadays, vasopressor-inotropes are used routinely in the management of various types of shock. Generalized vasoconstriction effects of these medications usually result in an increase in BP and therefore improve blood flow to hypo-perfused organs, but they may also cause a localized reduction in blood flow (peripheral ischemia) via excessive increase in blood vessel resistance, depending on which intermediate mechanism is predominant. Furthermore, there is considerable inter-individual variation in response to vasopressor-inotrope therapy. For instance, some patients can respond intensely even at low infusion rates. Therefore, all treatment related decisions in patients with CV compromise or shock necessitate an individualized approach to maximize the health benefit and minimize the risk for each patient.

Clinically, individualized treatment involves iteratively and empirically adjusting the infusion rate of an administered drug for each patient. In vasopressor-inotrope therapy, clinical guidelines include controlling BP as a measurable endpoint, with the goal of restoring effective tissue perfusion, namely volumetric blood flow. Therefore, it would be desirable to identify dose-response relationships in terms of both BP as well as blood flow, such that CO would designate the level of total blood delivery to the tissue, while BP would indicate when perfusion pressure is so low that even the vital organs, such as the heart, brain and adrenal glands, are likely hypo-perfused. Conversely, it would be desirable to identify BP values raising the risk of undue physiologic stress on the heart, resulting in decreased CO and organ blood flow. Such information would then be used to optimize the rate of vasopressor-inotrope infusion. Indeed, some techniques have been proposed to identify patient-specific dose-responses based on CO measured directly during vasopressor-inotrope dose changes. Such individualized approaches are helpful in light of substantial inter-individual dose response variability. However, these approaches are not truly viable in clinical practice since they rely on CO measurements which, at least during the early stabilization of patients with circulatory shock, are not readily available to infer complete CV state and individualized dose response relationships, in accordance with the present disclosure.

Vasoactive medications are used to treat hemodynamic compromise caused by CV pathology, e.g. shock (in which case the drugs are administered to restore tissue perfusion). These drugs are typically classified into three broad categories depending on the predominant pathway of action: vasopressor-inotropes, inotropes and lusitropes, although strict distinction between them is often difficult due to multiple effects associated with some drugs. Vasopressor-inotropes increase BP primarily by modulating vasoconstriction, namely TPR, and enhancing myocardial function. Specifically, inotropes improve myocardial contractility (and thus SV) and HR, resulting in an increase in CO and BP in most types of cardiogenic shock, while lusitropes cause vasodilation and improve cardiac function, especially in diastole period of the cardiac cycle. Most of these drugs function through stimulating the CV adrenergic receptors. The common adrenergic receptors associated with vasoactive drugs are alpha-adrenergic, beta-adrenergic, and dopaminergic receptors.

In particular, alpha-adrenergic receptors can be classified into two categories, namely α₁ and α₂. The α₁ receptors are found within the pre and post-synaptic regions in the sympathetic nerve endings on smooth muscle cells, and are also scarcely located on the myocardial cells. These receptors elicit several CV responses, including vasoconstriction of arteries and veins. Stimulation of the α₁ receptors in the vascular smooth muscle leads to vasoconstriction, resulting in an increase in BP via an increase in TPR. Also, these receptors are known to be responsible for an increase in the cardiac contractility at lower HR, although the relevant mechanism is still not clearly understood. On the other hand, beta-adrenergic receptors, namely β₁ and β₂, are located within the myocardium, although β₂ receptors are also found in the vascular and bronchial smooth muscles. The activation of β₁ adrenergic receptors exerts positive inotropic (increase in SV) and chrono- and dromotropic (increase in HR and conduction velocity, respectively) effects, both resulting in an increase in CO under appropriate conditions. The activation of β₂ receptors mediates smooth muscle relaxation so that a decrease in TPR occurs through the vasodilation of arteries. The hemodynamic effects of different vasoactive drugs depend on the subtypes and locations of the receptors it acts upon and the status of dynamic regulation of receptor expression in the CV system. For example, in the case of epinephrine, the α₁, β₁ and β₂ receptors are most relevant.

In some implementations, the hemodynamic responses to vasopressor-inotropes may be modeled in two layers. In the first layer, the relationships between the vasopressor-inotrope dose and the cardinal CV parameters (SV and TPR) and HR may be constructed using phenomenological models. In the second layer, the dependence of the BP on the cardinal CV parameters may be represented by the two-parameter model. This way, the cardinal CV parameters and HR included in the WK model can reproduce the inotropic, vasoactive and chronotropic effects of vasopressor-inotropes via their dependence on the dose of the medication. As described with reference to Eqn. 4, SBP and MAP can be fully characterized by two parameters, namely RC and

$$\frac{δ V}{C} .$$

Therefore, SBP and MAP response to vasopressor-inotrope dose can be predicted if the drug dependences of RC and

$$\frac{δ V}{C}$$

are known. Conversely, RC and

$$\frac{δ V}{C}$$

can be inferred from the observations of SBP and MAP values, although these cardinal CV parameters cannot be directly measured. It is well known that AC is inversely proportional to BP, and significantly correlated with age, SBP, PP, SV and CO. In some aspects, AC may be modeled as a function of SBP, namely

$$C = \frac{1}{{k}_{1} {P}_{s} + {k}_{2}} . \qquad ( 9 )$$

When the vasopressor-inotrope dose is administered, a quick increase in the drug's plasma concentration occurs initially. As the administration continues, the plasma concentration stabilizes at an equilibrium value through the balance between drug uptake, distribution and excretion. The medication thus distributed in the plasma is also distributed to the sites of action, namely the adrenergic receptor sites, with a phase lag.

Traditional approaches to modeling the drug distribution and equilibration process involves using the multi-compartmental model, where the body is divided into distinct “compartments” representing different classes of tissues. The equilibration of the drug between the plasma and the site of action has been modeled as a first-order phase lag or a first-order time-delayed system. More recently, a new direct dynamic dose-response model was proposed to describe the dose-dependent endpoint responses in case the measurement of the plasma drug concentration is not readily available. In some implementations, this approach can be used to model the dynamics related to the distribution and equilibration of vasopressor-inotrope in the plasma and the site of action as a first-order time-delayed system:

τ_{θ}\dot{θ}(t)+θ(t)=u(t−t_{D,θ}) (10)

where θε{θ_{R}, θ_{V}, θ_{H}} is the hypothetical vasopressor-inotrope dose at the site of action responsible for the changes in TPR (θ_{R}), SV (θ_{V}), and HR (θ_{H}), where τ_{θ} and t_{D,θ} are the time constant and the delay, respectively, dictating the phase lag associated with the distribution and equilibration of the vasopressor-inotrope, and u denotes the vasopressor-inotrope dose administered intravenously.

In some aspects, endpoints, or cardinal CV parameters, relevant to a vasopressor-inotrope may be assumed to be affected essentially by the hypothetical vasopressor-inotrope dose at the sites of action, namely the adrenergic receptor sites responsible for the respective CV parameters. As mentioned, the inotropic, chronotropic and vaso-constrictive effects of a vasopressor-inotrope are subject to actions from multiple receptors. Specifically, SV is affected by the α₁ and β₁ receptors, TPR is affected by the α₁ and β₂ receptors, and HR is affected by the β₁ receptor, represented generally as follows:

δV=δV₀+δV|_{β}_{₁}+δV|_{α}_{₁}

R=R₀+R|_{β}_{₂}+R|_{α}_{₁}

H=H₀+H|_{β}_{₁} (11)

where δV₀, R₀ and H₀ are baseline values of the respective CV parameters. The model dictating the effect of each receptor subtype on the cardinal CV parameters in Eqn. 11 is vasopressor-dependent.

By way of example, phenomenological models may be used to reproduce the responses of cardinal CV parameters in Eqn. 11 to the vasopressor-inotrope drug epinephrine, although it may be appreciated that other models, in dependence of the selected drug and mechanisms involved may also be utilized. Specifically,

$$\begin{matrix}\begin{matrix}δ V = δ {V}_{0} + δ V {{\vert}_{{β}_{1}} + δ V \vert}_{{α}_{1}} \\ = δ {V}_{0} + \underbrace{log ( {k}_{1 V} {θ}_{V} + 1 )}_{δ V {\vert}_{β1}} + \\ \underbrace{\{ - {( {k}_{2 V} {θ}_{V} + {k}_{3 V} )}^{{k}_{4 V}} \cdot {{θ}_{V}^{{k}_{5 V}} ( {θ}_{V}^{{k}_{5 V}} + {k}_{6 V} )}^{- 1} \}}_{δ V {\vert}_{{α}_{1}}}\end{matrix} & ( 12 a ) \\ \begin{matrix}R = {R}_{0} + R {{\vert}_{{β}_{1}} + R \vert}_{{α}_{1}} \\ = \frac{1}{\underbrace{{k}_{1 R} {θ}_{R} + {R}_{0}}_{{R}_{0} + R {\vert}_{β2}}} + \underbrace{{( {k}_{2 R} {θ}_{R} + {k}_{3 R} )}^{{k}_{4 R}} \cdot {{θ}_{R}^{{k}_{5 R}} ( {θ}_{R}^{{k}_{5 R}} + {k}_{6 R} )}^{- 1}}_{R {\vert}_{{α}_{1}}}\end{matrix} & ( 12 b ) \\ H = {H}_{0} + H {\vert}_{{β}_{1}} = {H}_{0} + {\underbrace{{k}_{1 H} {θ}_{H}^{k2H} ( {θ}_{H}^{{k}_{2 H}} + {k}_{3 H} )}_{H \vert {β}_{1}}}^{- 1} & ( 12 c )\end{matrix}$$

Epinephrine is a potent stimulant of both α and β adrenergic receptors, and its effect on MAP may be different depending on the administered dose. For sufficiently high epinephrine doses, an increase in MAP can be observed based on the following mechanisms: (i) increase in SV via positive inotropic action (β₁), which also results in an increase in SBP, (ii) increase in HR via positive chronotropic action (β₁), which, together with the increase in SV, results in an increase in CO, and (iii) increase in TPR via vasoconstriction (α₁), which then primarily increases DBP. Normally, the increase in SBP is greater than its DBP counterpart, and as a consequence, an increase in PP in proportion to the epinephrine dose can be observed. On the other hand, the effect is quite distinct if low doses of epinephrine are administered. For low epinephrine doses, the effect of β₂ action is more predominant than its α₁ counterpart, thereby resulting in peripheral vasodilation (primarily in the muscles). This, in turn, reduces DBP. Therefore, the MAP response may be subject to the changes in SBP and DBP. That is, MAP may increase, decrease, or remain constant. In addition, the effect on CO may further modify the BP response to low doses of epinephrine.

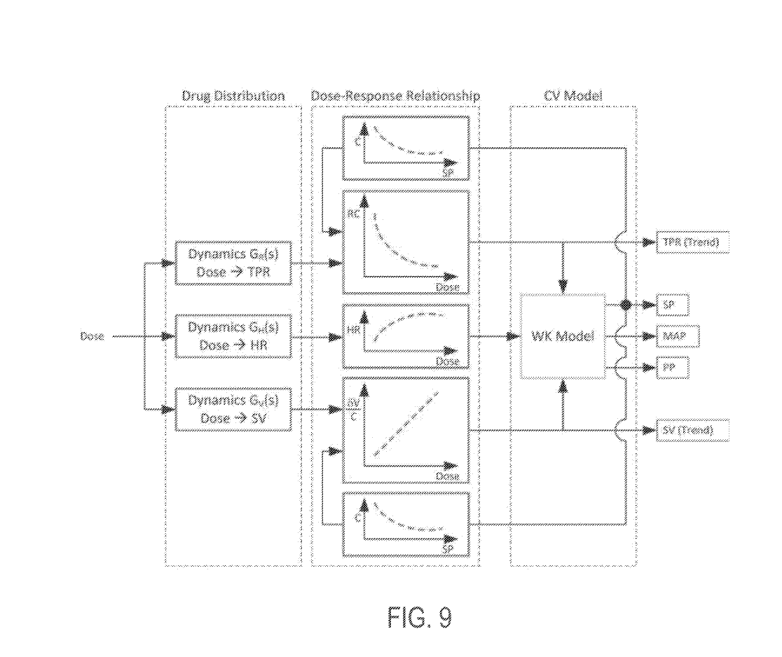

Based on the mechanisms of action with which epinephrine affects hemodynamic response, the phenomenological model associated with Eqns. 12 may be utilized to reproduce the effects of epinephrine on the various CV parameters (i.e., SV and TPR) and HR, where θ_{R}, θ_{V} and θ_{H} are the hypothetical vasopressor-inotrope doses at the sites of action (Eqn. 10), and k_{iV}, k_{iR}, and k_{iH} (i=1, . . . , 6) are unknowns to be determined. In order to reproduce the peripheral α₁ adrenergic effects on SV (i.e., decrease in SV due to excessively large afterload) and TPR (i.e., vasoconstriction) at high epinephrine doses, the Hill equation model may be introduced in the phenomenological models of SV and TPR to limit the α₁ adrenergic effects to the high-dose epinephrine region, with ^{k}^{_{5V}}\sqrt{k₆ᵥ}and ^{k}^{_{5R}} \sqrt{k_{6R}}being the level of epinephrine doses where the α₁ adrenergic effects on SV and TPR attain 50% of the maximum effects. Overall, the phenomenological models of Eqns. 12 are able to faithfully reproduce the critical behaviors of the cardinal CV parameters, as described below. Specifically, this approach can account for increase in SV in proportion to θ_{V} according to the β₁ adrenergic effect, with the rate of increase tapering off, or even decrease, in the high-dose region due to the large afterload caused by the α₁ adrenergic effect. Also a decrease in TPR in proportion to θ_{R} according to the β₂ adrenergic effect in the low-dose region, followed by the α₁ adrenergic effect which, when combined with its β₂ counterpart, can result in a further decrease in TPR with slower rate, a constant TPR (through the balance between β₂ and α₁ effects), or an increase in TPR (due to a strong vasoconstriction) can be achieved. Furthermore, an increase in HR in proportion to θ_{H} through the β₁ adrenergic effect, with the saturation occurring in the high-dose region, can also be achieved. The combined CV plus phenomenological model is visually shown in FIG. 9, where the dynamics of drug distribution are given by Eqn. 10, namely:

$${G}_{θ} ( s ) = {e}^{- {t}_{D , {θ}^{s}}} \frac{1}{{τ}_{θ} s + 1} \qquad ( 13 )$$

and the dose-response relationships are given by Eqns. 11 and Eqns. 12, and the WK model given by Eqns. 4.

To examine the validity of the above-described phenomenological model, experimental epinephrine dose-response data were collected from animal subjects. Five neonatal Yorkshire Duroc piglets of 10+/−3 d of age weighting 2.4+/−0.6 kg were pre-anesthetized with ketamine (33 mg/kg) and atropine (0.05 mg/kg), intubated, and anesthetized using 1.5-3.0% isoflurane while mechanically ventilated. Core body temperature was kept at 38+/−0.5° C. BP, HR, arterial O₂ saturation, end-tidal CO₂, fraction of inspired O₂, and pulmonary compliance were continuously monitored. HR beyond 250 bpm was captured using the PC-Vet wireless ECG system or direct counting. The animals received intravenous heparin (200 units/kg/h), physiologic saline (10 mL/kg/h), and 10 g/dL of dextrose-water to adjust serum glucose levels using Genie Plus syringe pumps validated for stability of infusion rate. Serum electrolytes, base excess (−6 to +6), pH (7.20-7.45), partial arterial O₂ pressure (80-120 mmHg) and partial arterial CO₂ pressure (35-45 mmHg), and arterial O₂ saturation (90-100%) were maintained in the target ranges using sodium bicarbonate, potassium chloride, lactated Ringer's solution, and ventilatory changes, respectively. Both femoral veins and left femoral artery were cannulated for fluid and drug administration, and arterial BP measurements and blood sampling, respectively. Changes in the hemodynamic parameters and serum electrolyte, lactate, and glucose and Hb concentration were followed while the dose of epinephrine was escalated. The baseline measurements were recorded after a 15-min postsurgical stabilization period, followed by the escalation of dose, and a 15-min washout period after the discontinuation of the medication. Each epinephrine dose was given for 15 min, where the last 5 min of each dose block yielded the equilibrium of the effects at each dose most accurately. To deduce the rate of change of the hemodynamic parameters, the data obtained were subdivided into baseline, low dose (0.25 mcg/kg/min), medium dose (0.5 and 0.75 mcg/kg/min) and high dose (1, 1.5 and 2 mcg/kg/min). BP and HR were collected in real time at a sampling rate of 0.2 Hz.

As described, the cardinal CV parameters (i.e., SV and TPR) were inferred from the BP, and the phenomenological models in Eqns. 12a and 12b together with the AC in Eqn. 9 were then tuned to the inferred responses of the cardinal CV parameters. In addition, the model of HR of Eqn. 12c was tuned directly based on acquired measurements. Specifically, the epinephrine dose and the associated BP (SBP and MAP) and HR were extracted from the experimental data. Then the estimates of the cardinal CV parameters (SV and TPR) were derived as follows. First, the estimates of

$$\frac{δ V}{C}$$

and RC were derived according to Eqns. 4 using the measurements of SBP, MAP and HR. Second, the phenomenological models in Eqns. 12a and 12b were combined with the AC given by Eqn. 9 to construct the following models:

$$\begin{matrix}\frac{δ V}{C} = \{ δ {V}_{0} + δ V {{\vert}_{{β}_{1}} + δ V \vert}_{{α}_{1}} \} \cdot ( {k}_{1} {P}_{s} + {k}_{2} ) & ( 14 a ) \\ RC = \frac{\{ {R}_{0} + R {{\vert}_{{β}_{1}} + R \vert}_{{α}_{1}} \}}{( {k}_{1} {P}_{s} + {k}_{2} )} & ( 14 b )\end{matrix}$$

where δV|_{β}_{₁}, δV|_{α}_{₁}, R|_{β}_{₂} and R|_{α}_{₁} are defined in Eqns. 12a and 12b. Third, the phenomenological models were fitted to the estimates of

$$\frac{δ V}{C}$$

and RC derived from the experimental BP and HR response data as follows:

$$K = arg {min}_{K} J ( u , {P}_{s} , {P}_{m} , H , K ) \qquad ( 15 )$$

where K [symbol] {τ_{θ}, t_{D,θ}, δV₀, R₀, H₀, k_{iV}, k_{iR}, k_{jH}, kₗ} denotes the set of unknown parameters, θε{θ_{R}, θ_{V}, θ_{H}}, i=1, . . . , 6, j=1, . . . , 3, l=1, 2, J=J_{V}+J_{R}+J_{H}, and

$$\begin{matrix}{J}_{V} ( u , {P}_{s} , K ) = {w}_{v} ‖ \frac{δ V}{C} - \{ δ {V}_{0} + δ V {{\vert}_{{β}_{1}} + δ V \vert}_{{α}_{1}} \} \cdot ( {k}_{1} {P}_{s} + {k}_{2} ) ‖ & ( 16 a ) \\ {J}_{V} ( u , {P}_{s} , K ) = {w}_{R} ‖ RC - \frac{\{ {R}_{0} + R {{\vert}_{{β}_{2}} + R \vert}_{{α}_{1}} \}}{( {k}_{1} {P}_{s} + {k}_{2} )} ‖ & ( 16 b ) \\ {J}_{H} ( u , K ) = {w}_{H} ‖ H - {H}_{0} - H {\vert}_{{β}_{1}} ‖ & ( 16 c )\end{matrix}$$

In formulating Eqns. 16 in each iteration of the optimization, the endpoints

$$\frac{δ V}{C} ,$$

RC and H were predicted using the epinephrine dose and SBP as inputs for a set of assumed model parameters, and the Euclidean norm of the difference between the observed and the predicted endpoints was calculated to evaluate the cost function J. The weights w_{V}, w_{R}, and w_{H} were chosen so that the magnitudes of J_{V}, J_{R} and J_{H} were comparable. The optimization problem in Eqn. 15 was solved by the Differential Evolution algorithm, which is a derivative-free optimization method suited for problems with real-valued, multi-modal and continuous-valued cost functions.

In solving the optimization in Eqn. 15, the uniqueness in the scales of δV, R and C was secured by assigning the baseline observations to δV₀ and R₀ in Eqns. 14. In addition, some of the parameters in K were constrained according to the physiologic relevance. First, all the parameters were constrained to assume positive values. Also, 0<k_{4V}, k_{4V}<1 was assumed to prevent the unboundedness of the α₁ adrenergic effects on SV and TPR in the high epinephrine dose region. Once the optimization was completed, the phenomenological expressions for δV, R and C were obtained using Eqns. 14.

Noting that the current dosing strategy for vasoactive drug therapy involves titrating the drug to keep the MAP above a pre-specified minimum, the ability to predict the BP response to vasoactive drugs can be extremely important to optimize the therapy. Using the model described, (FIG. 9) the BP (SBP and MAP) response to epinephrine administration can be predicted in the following way once the models are tuned to an individual. First, the hypothetical epinephrine doses at the sites of action can be predicted using Eqn. 10, using the epinephrine dose as input. Second, the cardinal CV parameters (SV and TPR) and HR can be predicted by the phenomenological models in Eqns. 12 with the dose at the sites of action as input. Third, AC can then be predicted by the SBP, which can be predicted from the SV, TPR and AC in the previous time step, where the initial value of SBP can be assumed to be available. Fourth,

$$\frac{δ V}{C}$$

and RC are calculated. Fifth, the WK model in Eqns. 4 may then be used to predict BP.

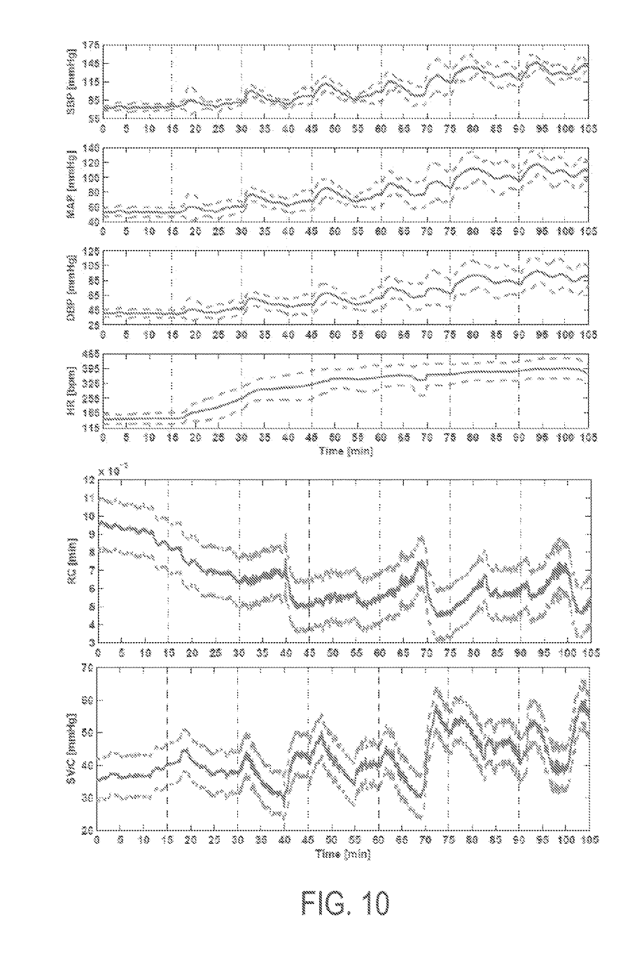

The hemodynamic responses averaged over the five piglets are illustrated in FIG. 10, which includes SBP, MAP, DBP and HR, as well as

$$\frac{δ V}{C}$$

and RC. It can be observed that BP (especially MAP) increases even in the low-dose region of epinephrine. In general, DBP decreases due to the vasodilatory effects of the β₂ adrenergic receptors at low epinephrine doses. It also appears that in the piglets studied, the vasodilatory effect may have been compensated by the increase in CO (through the significant increase in HR) and, to a minor extent, the decrease in AC. In addition, in the immature CV system, α receptor expression predominates and β receptor expression lags behind.

Similarly for MAP, a visible increase in MAP can be typically seen in the high epinephrine dose range where TPR starts to significantly increase via the α₁ adrenergic effect (vasoconstriction). By contrast, MAP persistently increases from the low-dose range in the data, as appreciated from FIG. 10. This can also be attributed to the increase in CO (again, through HR) that occurs in the relatively low and medium dose ranges, which appears to dominate the decrease in TPR in these dose ranges. In fact, HR increase is so significant that it attains 55% of its maximum response after the first dose escalation (0.25 mcg/kg/min). It appears that this large initial rise in HR leads to the rise in MAP through CO in the relatively low and medium epinephrine dose regions, even in the presence of vasodilation.

In regards to the cardinal CV parameters of SV and TPR, their trends cannot be clearly inferred from FIG. 10 since they are lumped with the effect of AC. In theory, RC is expected to decrease in the low dose range (since both TPR and AC are supposed to decrease), with the rate of decrease tapering off in the high dose range as the α₁ adrenergic effect (vasoconstriction) emerges, whereas

$$\frac{δ V}{C}$$

is expected to keep increasing (since SV is known to increase with epinephrine, and AC is known to decrease with the increase in SBP). The trends of RC and

$$\frac{δ V}{C}$$

are indeed consistent with the theoretical expectation, which supports the validity of using the CV model to infer cardinal CV parameters. Below, the efficacy of the implemented CV-phenomenological model to reproduce vasopressor-inotrope responses is illustrated using the results of system identification and prediction of BP responses associated with individual subjects.

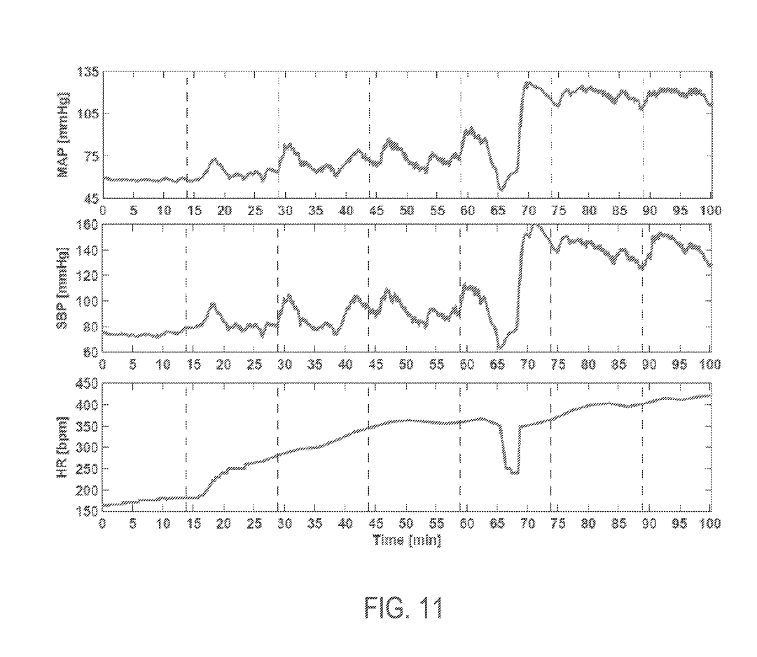

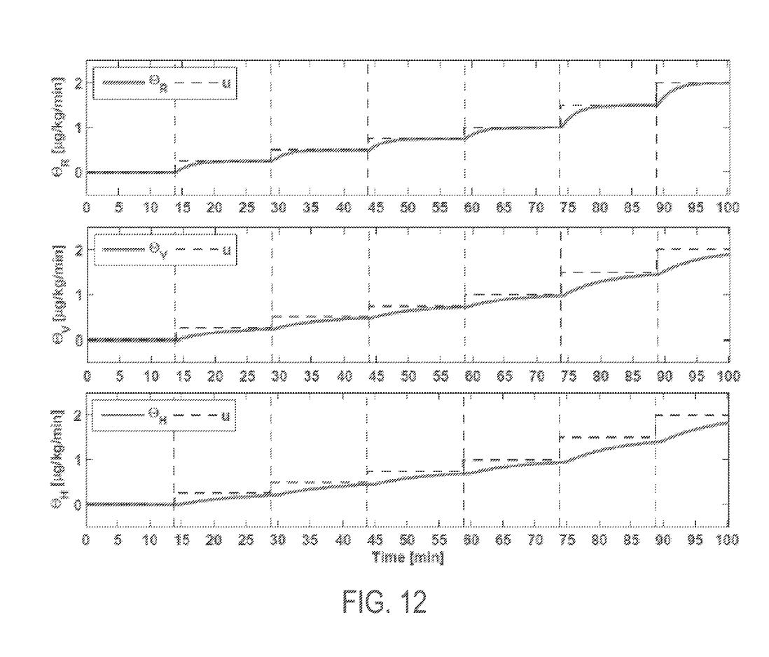

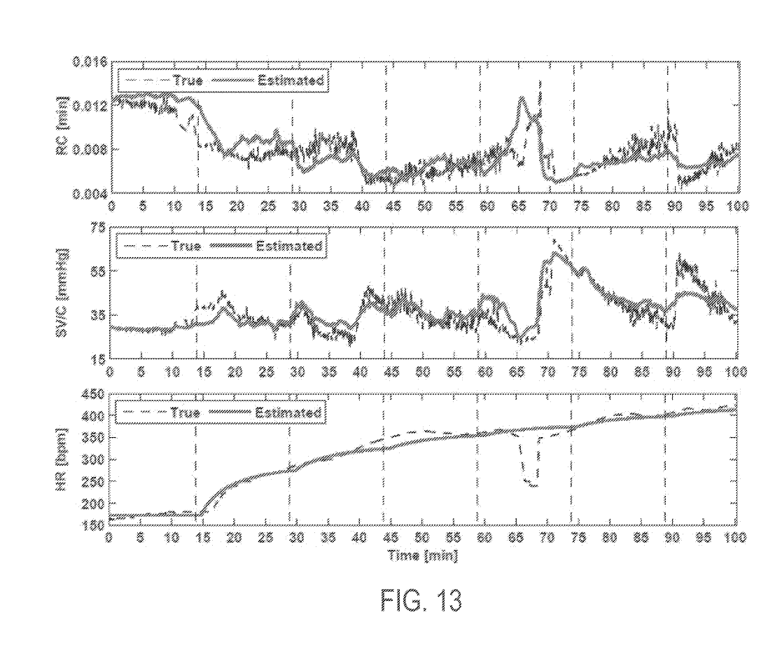

Overall, the optimization problem in Eqn. 15 was feasible to solve, and the resulting CV and phenomenological models were able to reproduce the hemodynamic responses adequately. FIG. 11 shows an example of BP and HR responses from a piglet for escalating epinephrine doses. BP and HR were used to carry out system identification in order to derive the phenomenological models in Eqns. 15 and the model of AC in Eqn. 12. Turning to FIG. 12, the intravenous versus hypothetical doses (at the sites of action) of epinephrine are shown. It can be found that for this particular piglet, the β₁ adrenergic effects (SV and HR) were determined to be slower than their β₂/α₁ counterparts, which is somewhat consistent with the endpoint observations in FIG. 13. Indeed, this may be appreciated by comparing the speed of responses of TPR and HR at the time instants when the epinephrine dose changes. In contrast, the speed of responses between TPR and SV was not clearly discernible, perhaps due to the fact that both RC and

$$\frac{δ V}{C}$$

involve AC, which was assumed to be a function of SBP in this study, and SBP is subject to change once any of SV, TPR or HR is altered. Therefore, the behavior of

$$\frac{δ V}{C}$$

was dominated by both the relatively slow response due to the β₁ adrenergic effect (i.e., increased cardiac contractility) and the relatively fast response due to the changes in SBP caused by the increase in TPR.

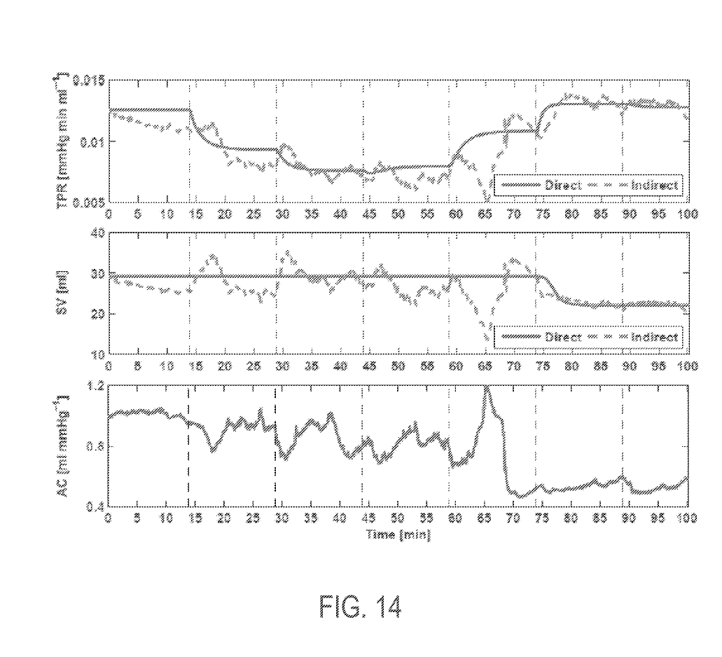

The models of the endpoints of interest (SV, TPR and HR) in Eqns. 12 and the AC in Eqn. 9 could faithfully reproduce the respective observations, as appreciated from FIG. 13. It is noted that the sudden drop in the HR response and the associated transients shown in SV and TPR responses at around 65 min is likely an artifact. Since the model of AC is available, the models of SV and TPR can be easily extracted from Eqns. 14 to obtain Eqns. 12a and 12b. It is, therefore, possible to estimate SV and TPR directly from the measurements of epinephrine dose and SBP. Alternatively, SV and TPR can also be estimated indirectly via the WK model, in which Eqn. 14a and Eqn. 9 are substituted into Eqn. 4a to estimate TPR, and Eqn. 14b and Eqn. 9 are substituted into Eqn. 4a to estimate SV. FIG. 14 is a graphical illustration comparing direct versus indirect estimates of SV and TPR, which are highly consistent with each other except for the fluctuations involved in the indirect estimate due to MAP, as appreciated from Eqn. 4a.

The response of TPR shown in FIG. 14 is consistent with the general knowledge that there is a decrease at low epinephrine doses due to vasodilation (i.e., β₂ adrenergic effect) but increases as the α₁ adrenergic effect starts to overcome its β₂ counterpart in the high epinephrine dose range. Considering the response of SV, this parameter is expected to increase as the afterload is reduced by the decrease in TPR. However, it appears that in this particular piglet, the initial HR increase is large enough to prevent SV from increasing by decreasing ventricular filling time. The result is the relatively constant SV in the low to medium epinephrine dose range. For higher doses of epinephrine, on the other hand, SV is reduced by the increase in TPR, which increases the afterload acting on the heart. Together with the saturating trend of HR response in the high dose range (FIG. 13), where for doses of 1-2 mcg/kg/min, HR starts to saturate at around 400 bpm, a decrease in CO is anticipated. In fact, this model-based inference is highly consistent with the previously performed direct analysis of the same data.

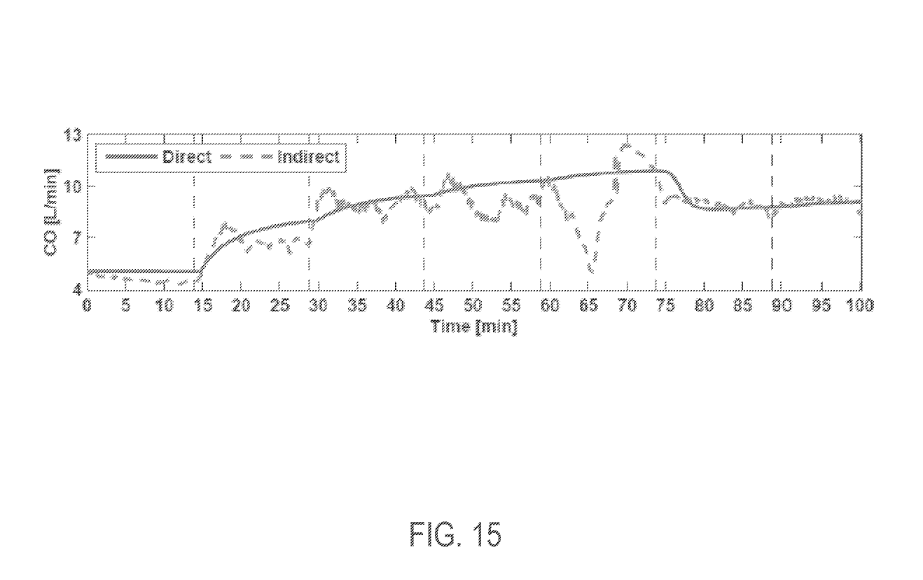

The CO estimated by the identified models indeed predicts that CO would decrease at higher doses of epinephrine, as shown in FIG. 15. It should also be mentioned that the initial increase in CO in the low to medium epinephrine dose range, responsible for the increase in MAP, is also predicted by the models accurately, as shown in FIG. 15. The aforementioned findings on the behavior of the proposed CV and phenomenological models were also valid for all the piglets studied to a large extent, further supporting the validity of the present approach to reproduce the hemodynamic responses to vasopressor-inotropes. The identified model parameters are summarized in terms of mean and standard deviation in Tables 2 and 3.

TABLE 2
Pharmacokinetic and pharmacodynamic parameters of cardiovascular parameters.

| X | l/ [symbol] | t [symbol] [sec] | X₀ | k_{1X} | k_{2X} | k_{3X} | k_{4X} | k_{5X} | k_{6X} |
|---|---|---|---|---|---|---|---|---|---|
| [symbol] V | 0.01 ± 0.02 | 29.1 ± 26.1 | 33.4 ± 6.91 | 2.3e3 ± 2.5e3 | 0.64 ± 1.60 | 4.3e3 ± 8.8e3 | 0.49 ± 0.28 | 25.1 ± 31.9 | 14.3 ± 35.0 |
| R | 0.03 ± 0 05 | 48.6 ± 17.6 | 76 4 ± 56.7 | 96 8 ± 33 9 | 7.7e−4 ± 1.1e−3 | 4.0e−3 ± 3.1e−3 | 0.96 ± 0.07 | 31.1 ± 36.2 | 14.7 ± 34.8 |
| H | 4.1e−3 ± 4.5e−3 | 25.1 ± 24.8 | 155 ± 24 3 | 239 ± 55 3 | 4.05 ± 3.70 | 0 [symbol] 18 ± 0.27 | — | — | — |

[symbol]

indicates data missing or illegible when filed

It can be readily observed that the parameters exhibit significantly large inter-individual variability, which in turn suggests the necessity to make individualized predictions of vasopressor-inotrope responses.

TABLE 3
Arterial compliance parameters.

| k₁ | k₂ |
|---|---|
| 1.3e−2 ± 7.8e−2 | 2.2e−3 ± 1.8e−1 |

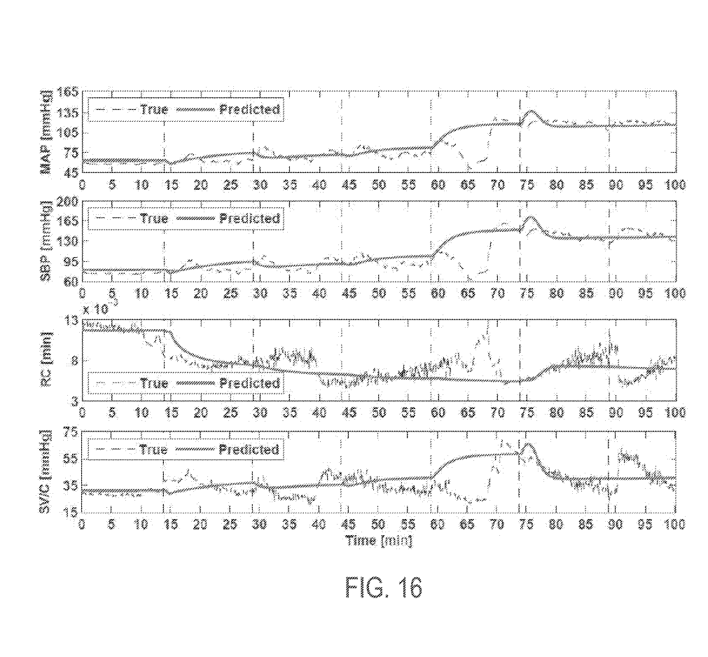

FIG. 16 shows the hemodynamics predicted using the CV and phenomenological model, individualized for the piglet of FIG. 11. It is clear that the BP (both SBP and MAP) were predicted with remarkable accuracy. In addition, the trends of RC and

$$\frac{δ V}{C}$$

could also be well predicted, although the details of the fluctuation were not resolved, due to the smoothly predicted SBP. Of note is that the actual SBP observation was used as input when estimating RC and

$$\frac{δ V}{C}$$

in FIG. 13, whereas in FIG. 16, the only input to the CV plus phenomenological models was the dose of epinephrine. The large transient prediction errors at 65 min were likely due to the artifact in the observations.

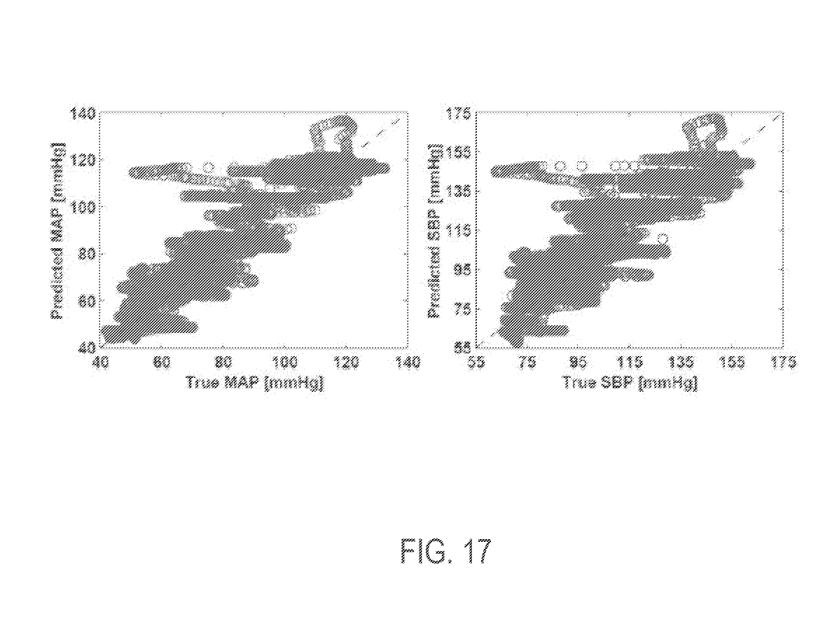

The prediction of hemodynamic responses was also carried out with the averaged model parameters listed in Tables 2 and 3. Although not shown, the predicted BP responses were divergent with large errors. This reassures the importance of the individualization in the prediction of hemodynamic responses to vasopressor-inotropes. The validity of the CV plus phenomenological models was quantified in terms of the r² value between actual and predicted SBP and MAP values. Table 4 shows that the r² values are consistently higher than 0.89 for predicting MAP and 0.85 for predicting SBP, respectively, strongly suggesting the efficacy of the present approach to modeling of vasopressor-inotrope responses, as illustrated in FIG. 17.

TABLE 4
R² values of blood pressure prediction.

| | Subject | | | | |
|---|---|---|---|---|---|
| | 1 | 2 | 3 | 4 | 5 |
| MAP | 0.92 | 0.94 | 0.89 | 0.89 | 0.91 |
| SBP | 0.90 | 0.91 | 0.85 | 0.85 | 0.86 |

In the above-described studies, CV and phenomenological models were used to reproduce the dose responses to the administration of pharmaceutical agents for treatment. As described, the uniqueness of the approach described lies in the capability of inferring cardinal CV parameters defining the CV state of a subject, which in many cases are unavailable from direct observations, in addition to the various clinical endpoints, such as BP responses. The feasibility of the models was illustrated using the experimental epinephrine response data, demonstrating potential for predicting responses for optimized therapy.

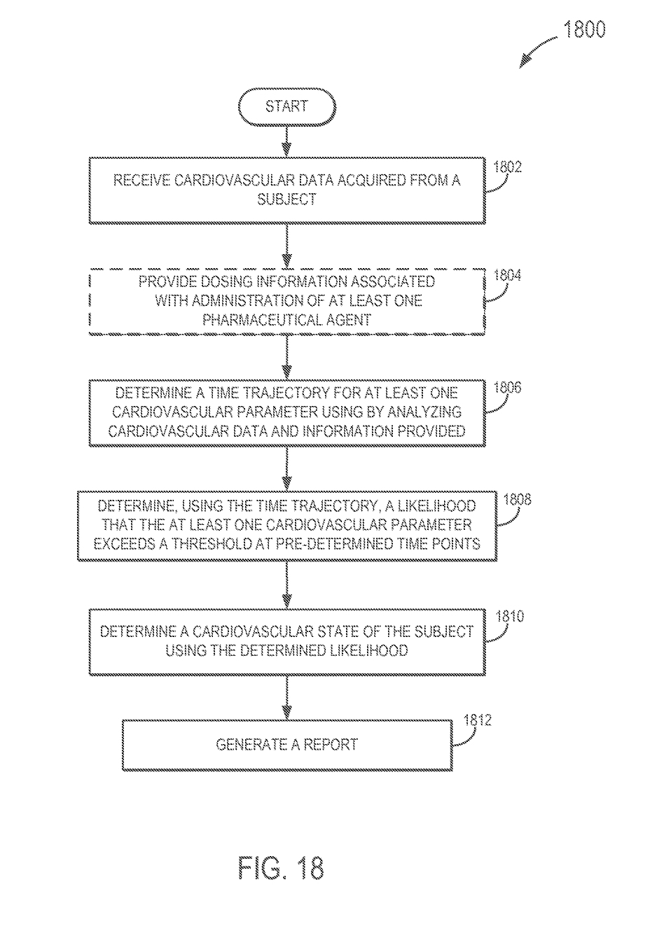

Turning now to FIG. 18, steps of another process 1800 for monitoring and/or controlling a CV state of a subject are shown. The process 1800 may begin at process block 1802 where CV data, and other physiological data associated with a subject may be received or acquired using a system, for example, as described with reference to FIG. 1, either continuously or intermittently using various sensors, such as BP sensors. Optionally, at process block 1804, a variety of information may also be provided. For example, dosing information associated with administration of at least one pharmaceutical agent, such as vasopressor data, as well as dose change event data may be provided. Such information may be provided by direct input by a user, such as via buttons, touch screens or other input elements, or may be obtained by communicating with various systems or devices, or treatment units, such as a programmable infusion pump. In some aspects, information may be retrieved from a memory or other storage location. For example, a subject's reference data, such as dose response data, and other data associated with the patient may be provided. Also, certain information associated with a subject may be determined automatically using changes in various measured quantities, such as BP or heart rate. In addition, dose responses, confidence intervals, and other data obtained from a population of subjects, may also be retrieved or provided.

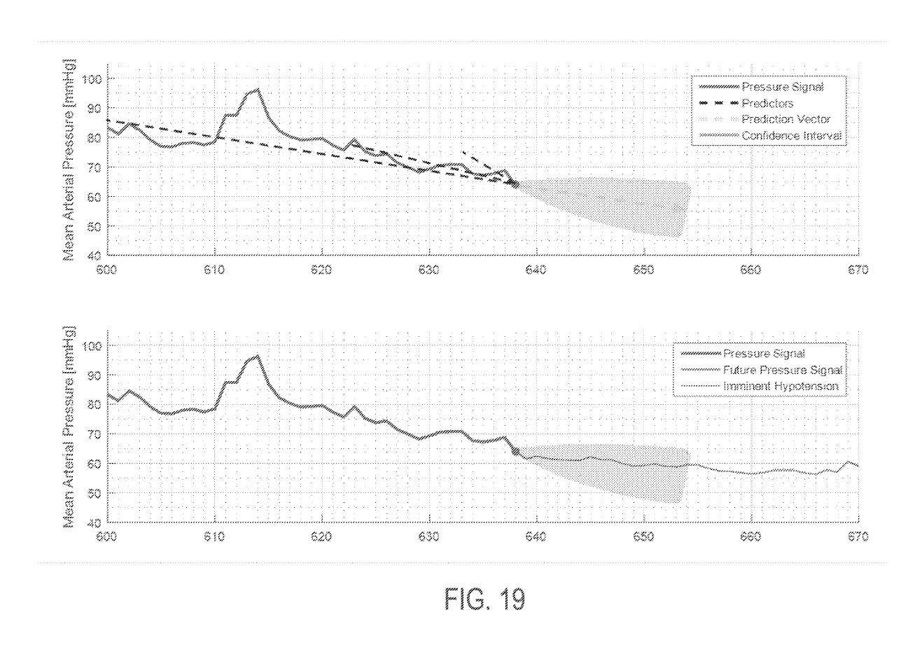

At process block 1806, a time trajectory for at least one CV parameter may be determined, by analyzing CV data and other information provided. Then, at process block 1808, may be performed to determine a future CV state of a subject using a variety of inputted or determined information. In some aspects, a statistical analysis may be performed to determine the future CV. For example, various Monte Carlo simulations, regression model analyses, and other formulae that provide a statistical result based on predicted time trajectories, confidence intervals, dosing information, and other important clinical factors, may be utilized. In some aspects, a statistical model may be applied to determine probabilities or likelihoods that one or more CV parameters, such as BP, exceeds predetermined thresholds or ranges of values at one or more pre-determined time points. By way of example, FIG. 19 shows an example of time traces for MAP, illustrating predictors for determining a future BP, such as recent or long-term BP trends, as well as confidence intervals. In some aspects, recent trends or information may be weighed more heavily in performing the analysis.

As such, a future CV state or condition of the subject may be determined, using such determined likelihoods or probabilities, as indicated by process block 1810. In some aspects, a determination of a future CV state may include identifying whether a subject's BP will exceed one or more thresholds at particular points in time, or whether BP will persist outside of a selected range longer than a specified duration of time.

In some aspects, the future CV state may inform a determination for adjusting a dose of at least one administered pharmaceutical agent. This may include using a variety of information, such as dosing information, as well as user input. For example, information associated with a recent dose change would be informative in that a pharmacological effect would not have a change to take place, and hence a delay in adapting treatment may be desirable. As may be appreciated from the above description, this approach may be fully configurable. For instance, in managing BP, precise control may be achieved by selecting a narrow range for allowable BP values, while permissive control would imply wider goal range, with tolerance for brief excursions outside the range. In this manner, a number of clinician notifications may be minimized, resulting in fewer interruptions to clinician in the typical clinical setting.

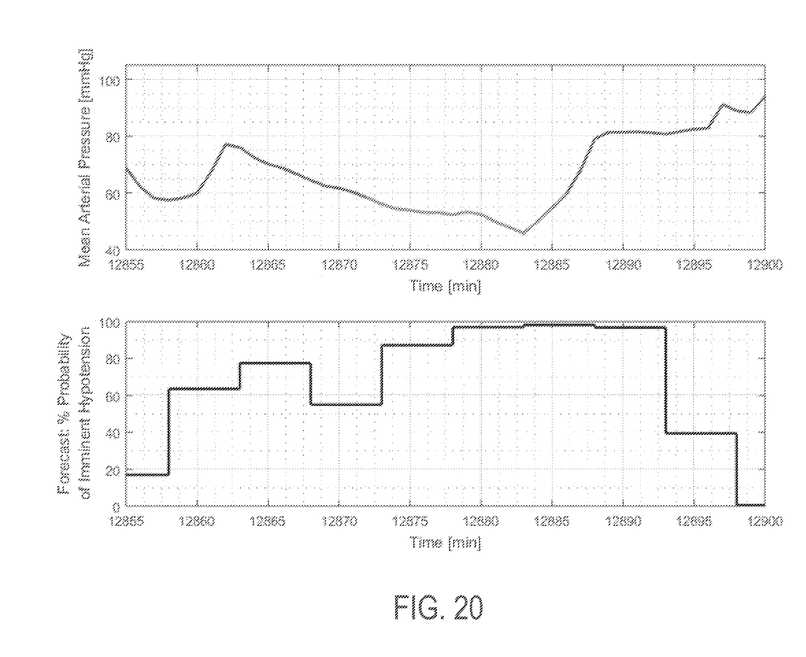

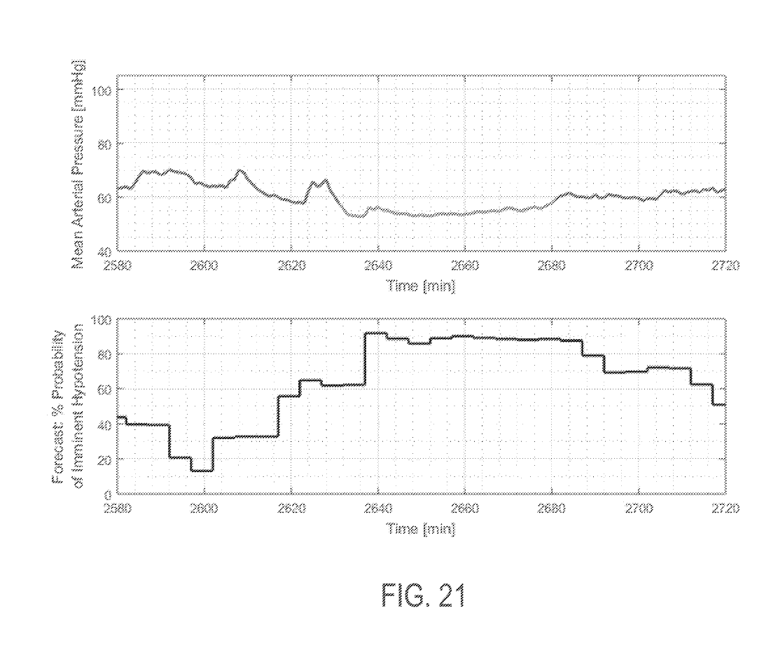

Then, at process block 1812 a report indicative of the future CV state of the subject may be generated. The report may be in any form, and include any information. In some aspects, the report may be in the form of an audio or visual notification provided to a clinician, for instance, indicating when particular CV parameters exceed selected ranges or thresholds. In addition, the report may indicate a risk or probability for a future CV state, such as an imminent or sustained risk for hypotension, hypertension, and so forth. By way of example, FIGS. 20 and 21 indicate examples of time traces for MAP, identifying high probability of imminent hypotension, correlated with measurements of lower MAP. In some aspects, a notification may be provided via a remote communication, such as a text message, or central display station.

In some aspects, the report may provide an indication to a clinician for modifying a treatment. Alternatively, or additionally, the report may generate an output for controlling a treatment unit dispensing one or more pharmaceutical agents. In this regard, steps may be carried out at process block 1812 for controlling the CV state of the subject at one or more time points, either by a clinician or a system configured to do so. This can include generating a CV model describing the CV state of the subject, and estimating dose responses associated with at least one administered pharmaceutical agent for one or more CV parameter. As described, this may include inferring parameter values using the BP data or the HR data, or both, acquired at two or more time points. Examples of CV parameters include BP, CO, TPR, AC, SV, and others.

In summary, the present disclosure provides systems and methods for monitoring and/or controlling a CV state of a subject using one or more administered pharmaceutical agent. In some aspects, present and potential future CV state of a subject are predicted in order to inform administration of at least one pharmaceutical agent, such as a vasopressor, by a clinician or a system suitable to do so. These can facilitate treatment in today's busy clinics, particularly for critically ill patients.

In particular, since current clinical practice involves manual dosage adjustments, there are times when inevitably dosage is not optimal and consequently the CV state of the patient is suboptimal. Using systems and methods described, optimized dosage changes may be provided, with minimal or no clinician input, in a way that can quickly move a patient to an optimal CV state, with minimal inadequate or excessive adjustments. Rapidly adapting to an optimal infusion rate would then lead to superior outcomes for critically-ill patients, because their organs will be spared from intervals of hypo-perfusion (i.e., infusion rate too low), and of excessive vasoconstriction with reduced perfusion (i.e., infusion rate too high). As such, the benefit of the present approach, over the subjective judgment of the clinician, might be especially pronounced for patients being managed by clinicians without extensive critical care expertise.

Also, it is envisioned that the present approach will enable a new generation of more sophisticated and more effective strategies for optimizing vasopressor dosages, as suggested by recent investigations. For instance, present guidelines for dose adjustment are quite vague. For example, the Surviving Sepsis campaign advises that, for patients with septic shock, clinicians infuse vasopressors to a MAP of at least 65 mmHg. This broad hemodynamic target has the advantage of its simplicity. However, there are varieties of CV states that satisfy such general criterion, creating situations when an improper or inadequate treatment is administered. For instance, in a patient with shock, the MAP could be elevated by organ vasoconstriction alone, which would improve essential perfusion to the brain and heart (these specific organs are relatively insensitive to vasopressors), but at the expense of perfusion to other vasoconstricted organs. In addition, in many cases, it might be preferable to optimize CO, and not just MAP, since improved CO could translate to improved blood flow to all the organs of the body. Moreover, excessive increases in MAP can cause strain on the heart, namely an increased oxygen requirement for the cardiac muscle tissues and the risk of reduced pump function. Increasing doses of vasopressors can likewise raise HR, which can also strain the heart. As such, it is envisioned that the present approach would be superior to today's coarse management strategies in that dose response relationships for various CV parameters allow for more sophisticated therapeutic strategies. In this manner, the trade-offs between various clinical goals or endpoints can be balanced. For example, optimized perfusion of the heart and brain (i.e., adequate MAP) can be achieved, while also obtaining perfusion of other organs (i.e., adequate CO) and cardiac work (i.e., non-excessive MAP and HR). An analytic tool or approach, as provided herein, that adapts to the individual patient and can accurately predicts the proper vasopressor infusion rate to achieve tight optimized targets for MAP and CO will make it practical to employ such more sophisticated therapeutic strategies. By contrast, today's clinician simply lacks the capability to do much more than adjust, and re-adjust the dose, to hit broad cardiac targets.

The present invention has been described in terms of one or more embodiments, including preferred embodiments, and it should be appreciated that many equivalents, alternatives, variations, and modifications, aside from those expressly stated, are possible and within the scope of the invention. As used in the claims, the phrase “at least one of A, B, and C” means at least one of A, at least one of B, and/or at least one of C, or any one of A, B, or C or combination of A, B, or C. A, B, and C are elements of a list, and A, B, and C may be anything contained in the Specification.

1. A system for monitoring a cardiovascular state of a subject, the system comprising:
at least one sensor configured to acquire cardiovascular data from a subject;
at least one processor configured to:
analyze the cardiovascular data to determine a time trajectory for at least one cardiovascular parameter;
determine, using the time trajectory, a likelihood that the at least one cardiovascular parameter exceeds a threshold at one or more pre-determined time points;
determine a future cardiovascular state of the subject using the determined likelihood;
generate a report indicative of the future cardiovascular state of the subject; and
an output for displaying the report to a user.

2. The system of claim 1, wherein the at least one processor is further configured to determine the likelihood using dosing information associated with administration of at least one pharmaceutical agent.

3. The system of claim 1, wherein the at least one processor is further configured to generate a cardiovascular model describing a cardiovascular state of the subject.

4. The system of claim 1, wherein the at least one processor is further configured to make a determination for adjusting a dose of the at least one administered pharmaceutical agent.

5. The system of claim 4, wherein the at least one processor is further configured to, based on the determination, estimate a dose response associated with at least one administered pharmaceutical agent for the at least one cardiovascular parameter.

6. The system of claim 4, wherein the at least one processor is further configured to control the future cardiovascular state of the subject at the one or more pre-determined time points using the estimated dose response.

7. The system of claim 1, wherein the at least one cardiovascular parameter includes at least one of a blood pressure, a pulse pressure, a systolic blood pressure, a diastolic blood pressure, and a mean arterial pressure.

8. A system for controlling a cardiovascular state of a subject, the system comprising:
at least one sensor configured to acquire cardiovascular data from a subject; and
at least one processor configured to:
receive the acquired cardiovascular data;
generate a cardiovascular model describing the cardiovascular state of the subject;
estimate a dose response associated with at least one administered pharmaceutical agent for at least one cardiovascular parameter defining the cardiovascular state;
control the cardiovascular state of the subject at one or more pre-determined time points using the estimated dose response.

9. The system of claim 8, wherein the cardiovascular data includes blood pressure data and heart rate data.

10. The system of claim 9, wherein the at least one processor is further configured to infer values for at least one cardiovascular parameter using the blood pressure data or the heart rate data, or both.

11. The system of claim 8, wherein the cardiovascular data is acquired at two or more time points.

12. The system of claim 8, wherein the at least one processor is further configured to determine, using the acquired cardiovascular data, a likelihood that at least one cardiovascular parameter defining the cardiovascular state of the subject exceeds a threshold at the one or more pre-determined time points.

13. The system of claim 8, wherein the processor is further configured to receive dosing information associated with administration of at least one pharmaceutical agent.

14. The system of claim 8, wherein the at least one cardiovascular parameter includes at least one of a blood pressure, a cardiac output, a total peripheral resistance, an arterial compliance, and a stroke volume.

15. A method for monitoring a cardiovascular state of a subject, the method comprising:
acquiring cardiovascular data from a subject using at least one sensor;
analyzing the cardiovascular data to determine a time trajectory for at least one cardiovascular parameter;
determining, using the time trajectory, a likelihood that the at least one cardiovascular parameter exceeds a threshold at one or more pre-determined time points;
determining a future cardiovascular state of the subject using the determined likelihood; and
generating a report indicative of the future cardiovascular state of the subject.

16. The method of claim 15, wherein the method further comprises determining the likelihood using dosing information associated with administration of at least one pharmaceutical agent.

17. The method of claim 15, wherein the method further comprises generating a cardiovascular model describing a cardiovascular state of the subject.

18. The method of claim 15, wherein the method further comprises making a determination for adjusting a dose of the at least one administered pharmaceutical agent based on the determined future cardiovascular state.

19. The method of claim 18, wherein the determination includes identifying whether a dose adjustment exceeds a pre-determined threshold.

20. The method of claim 18, wherein the method further comprises to estimating, based on the determination, a dose response associated with at least one administered pharmaceutical agent for the at least one cardiovascular parameter.

21. The method of claim 20, wherein the method further comprises controlling the future cardiovascular state of the subject at the one or more pre-determined time points using the dose response estimated.

22. The method of claim 15, wherein the at least one cardiovascular parameter includes at least one of a blood pressure, a pulse pressure, a systolic blood pressure, a diastolic blood pressure, and a mean arterial pressure.

23. A method for controlling a cardiovascular state of a subject, the method comprising:
receiving cardiovascular data acquired from a subject;
generating a cardiovascular model describing the cardiovascular state of the subject;
estimating a dose response associated with at least one administered pharmaceutical agent for at least one cardiovascular parameter defining the cardiovascular state; and
controlling the cardiovascular state of the subject at one or more pre-determined time points using the estimated dose response.

24. The method of claim 23, wherein the cardiovascular data includes blood pressure data and heart rate data.

25. The method of claim 24, wherein the method further comprises inferring values for at least one cardiovascular parameter using the blood pressure data or the heart rate data, or both.

26. The method of claim 23, wherein the cardiovascular data is acquired at two or more time points.

27. The method of claim 24, wherein the method further comprises determining, using the acquired cardiovascular data, a likelihood that at least one cardiovascular parameter defining the cardiovascular state of the subject exceeds one or more thresholds at the one or more pre-determined time points.

28. The method of claim 23, wherein the method further comprises making a determination whether at least one cardiovascular parameter defining the cardiovascular state of the subject exceeds the one or more thresholds for a selected duration.

29. The method of claim 27, wherein controlling the cardiovascular state of the subject includes adjusting a dose of the at least one administered pharmaceutical agent based on the determination.

30. The method of claim 23, wherein the method further comprises receiving dosing information associated with administration of at least one pharmaceutical agent.

31. The method of claim 23, wherein the at least one cardiovascular parameter includes at least one of a blood pressure, a cardiac output, a total peripheral resistance, an arterial compliance, and a stroke volume.
